(19) United States
(12) Patent Application Publication
(10) Pub. No.: US 2014/0271863 A1
(43) Pub. Date: Sep. 18, 2014

(54) HOT WASHABLE POLY-N-ISOPROPYLACRYLAMIDE HYDROGEL DELIVERY SYSTEMS

(71) Applicant: Textile-Based Delivery, Inc., Seattle, WA (US)

(72) Inventors: David Anderson, Rockford, MN (US); Jordan Schindler, Tucson, AZ (US)

(73) Assignee: TEXTILE-BASED DELIVERY, INC., Seattle, WA (US)

(21) Appl. No.: 14/213,808

(22) Filed: Mar. 14, 2014

Related U.S. Application Data
(60) Provisional application No. 61/785,081, filed on Mar. 14, 2013.

(51) Int. Cl. A61K 47/32; A61K 31/4458; A61K 9/06

(52) U.S. Cl. CPC A61K 47/32; A61K 9/06; A61K 31/4458
USPC 424/486; 514/330

(57) ABSTRACT

The present disclosure is in the field of controlled release delivery of actives (e.g., drug-delivery, cosmeceutics, alternative medicine) though from an unusual and novel delivery vehicle: personal clothing. Means are disclosed for producing controlled-release systems well-suited not only for affixation to fabrics—e.g., clothing, bedding—but also for maintenance of active throughout washing cycles including hot-wash laundry cycles. Some embodiments comprise a material comprising a unique combination of the thermoresponsive polymer poly-N-isopropylacrylamide, crosslinked and water-swollen in the form of a hydrogel, with embedded thin platelet-shaped solid crystals, drug-solubilizing microparticles, and a drug or other active that is released at ambient or body temperature, but only slowly in warm or hot water. Also disclosed are particles comprising solid dispersions of active compounds dispersed in nested or layered arrangements of at least two distinct polymers, in a fiber- or rod-shaped configurations, satisfying certain mathematical criteria.

### CROSS-REFERENCE TO RELATED APPLICATION

This application claims the benefit of U.S. Provisional Application No. 61/785,081, filed Mar. 14, 2013, the disclosure of which is hereby incorporated by reference herein in its entirety.

### BACKGROUND

The prior art contains many examples of polymer matrices that release active compounds such as drugs, nutriceuticals, cosmeceuticals, etc. It is essentially a universal feature of these materials that release of active remains the same, or much more commonly increases, with increase in temperature. This limitation poses the fundamental problem of hot water incompatibility, and with reusability after washing in water at any temperature, due to the large volumes of water in everyday washing regimens and/or rapid release of active from the delivery vehicle at typical washing temperatures.

U.S. Pat. No. 7,780,979, to Hu et al., involves fabric-supported chitosan modified temperature responsive poly-N-isopropylacrylamide/polyurethane hydrogel and the use thereof in preparation of facial mask. The increase in temperature of the hydrogel upon contact with the skin causes, in that invention, the deswelling of the hydrogel, which releases active from the hydrogel. In one embodiment, for example, the releasing rate at 37° was increased 2-3 times compared to that at 20° due to the high temperature-induced shrinkage.

U.S. Patent Application No. 2008/0057809, to Rock, describes a textile fabric having a smooth surface with one or more regions having a bound coating of hydrogel exhibiting expansion or contraction in response to change in relative humidity or exposure to liquid sweat or a combination thereof, adjusting insulation performance, air movement, and/or liquid management of the textile fabric in response to ambient conditions. The purpose of the thermoresponsiveness is to open up the fabric for increased air flow in response to sweating during exercise. Release of actives is not mentioned.

U.S. Patent Application No. 2009/0158492, to Yao, describes a quick-drying textile, in which poly-N-isopropylacrylamide or poly(propyleneoxide-co-ethyleneoxide) is incorporated to yield a material that changes water absorption properties with temperature, thereby reducing energy requirements for drying the textile.

U.S. Pat. No. 7,316,919, to Childs et al., discloses a composite material comprising (a) a support member comprising a plurality of pores extending through the support member; and (b) a non-self-supporting macroporous cross-linked gel comprising macropores having an average size of 10 nm to 3000 nm, said macroporous gel being located in the pores of the support member; wherein said macroporous cross-linked gel is present in the pores of the support member in an amount sufficient such that, in use, liquid passing through the composite material passes through said macropores of said macroporous cross-linked gel; and wherein said macropores of said macroporous cross-linked gel are smaller than said pores of said support member. The fluid-filled macropores thus have an average size greater than about 10 nm, and more preferably greater than about 25 nm, which is much larger than the micropores of a normal hydrogel, and in fact larger than the micropores of the hydrogels in that invention, since the macropores are produced by a process that generally involves the use of a porogen, i.e., a moiety of the desired macropore size that is removed after polymerization.

U.S. Pat. No. 7,713,440, to Anderson, discloses pharmaceutical compositions comprising a plurality of uncoated, ionically charged particles of a single thermodynamic equilibrium reversed cubic phase material, dispersed in a liquid comprising a polar solvent and stabilized in dispersion by said ionic charge, said particles having a size from 10 nm to 100 microns. The addition of solids and solutes to the exterior phase of such a dispersion is discussed in Anderson, but the patent teaches only dispersed particles in the fully hydrated state: “By incorporating a non-volatile additive in the exterior phase, preferably dissolved but alternatively dispersed, drying can result in particles that are kept from liquid crystal-liquid crystal fusion by the presence of an intervening solid . . . . Since the solid is either soluble, or readily dispersible, in the original liquid (usually water), then addition of this liquid will generally result in prompt reconstitution of a dispersion.”

U.S. Patent Application No. 2010/0092529, to Chetboun, purports that “an acaricidal and antimicrobial treatment for textile materials, e.g., for combating house dust mites, comprises spraying or padding with composition containing microencapsulated neem oil,” where neem oil is the fixed oil extracted from the plant Azadirachta indica. However, despite claims in popular promotional literature, there is no published scientific evidence that neem oil is antibacterial. The Chetboun disclosure makes a vague statement relating to diffusion of neem oil from the microcapsules: “It was also shown that the treated textiles are provided with an ability to transfer the agaricidal/mite-inhibiting properties by diffusion of the released active principles onto untreated textile materials in contact with a treated textile.” The rate of diffusion of the (relatively high-MW) compounds making up the neem oil was not given, and one skilled in the art would rightfully expect this rate to be extremely small for urea-formaldehyde capsules capable of sustaining active through multiple wash cycles. In any event, this release from one textile onto another appears oblique with regards to delivery of active to the skin or other tissue.

U.S. Patent Application No. 2011/0142898, to Fan, purportedly describes an “Article, useful for treating and preventing skin disorders, comprises a layer of fabric for contacting with the human skin, and an indicator attached on the fabric layer for indicating the condition of the layer.” However, absolutely no description or even suggestion as to how one would make, much less embed, an indicator of any sort is provided in that disclosure.

U.S. Pat. No. 5,299,335, to Ivester and Watson, states: “The primary object of this invention is to provide improved stuffed articles, such as a pillow, mattress, furniture cushion or the like, in which capsules impregnated with volatile substance(s) are secured internally within the article so as to prevent the capsules from migrating to location(s) substantially removed from those in which the capsules were placed during initial assembly of the stuffed article.” Thus, the objective is to prevent the capsules from migrating out of the secured, internal positions to the surface where, e.g., undesired direct contact with humans, particularly skin, could be encountered.

Zu Putlitz et al. [Adv. Mater. (2001) 13(7):500] reported “armored latexes” where a polymer nanoparticle is completely covered with clay plates or “scales,” and touted them as potential pressure sensitive adhesives.

U.S. Pat. No. 4,314,557, to Chandrasekaran, discloses a bandage that is designed for dissolution-limited release of a drug from the skin-contacting side of a flat polymer film containing dispersed drug. Of the two “major surfaces” of the thin film, one is occluded, and release of drug occurs through the entirety of the other “major surface.” In the adhesive bandages of that disclosure, the release through this major surface then continues through a drug-permeable adhesive layer that provides attachment to the skin. Mathematical conditions on the drug-polymer system are provided that ensure a near-constant rate of release. The rate of release of active is proportional to the surface area S of the “major surface,” which is much larger than the square (12) of the thickness of the film, and in fact 1 must be kept small in order to maintain the condition that 1K/D<0.06 required for near-constant release rate. Since the total duration of release under these dissolution-limited conditions is proportional to 1, U.S. Pat. No. 4,314,557 does not provide for independent control of release rate and duration of release to a given area of skin, particularly for long release times: weeks or months, not hours. Quite generally, a film format—with approximately 50% non-occluded surface area—is not a good format for achieving long duration of release because the high area (and thus rate) of release combines with the low fill volume (due to small thickness 1) to yield short durations. According to the U.S. Pat. No. 4,314,557 specification, the thickness will usually be in the range of 50 to 1,500 microns (0.005 to 0.15 cm); thus, a bandage of approximately 1 sq. inch would have a thickness-to-diameter aspect ratio 1/d of less than 0.1, where d is the diameter for the case of a circular bandage, or characteristic width of a square bandage, etc.

### SUMMARY

The limitations of the prior art reviewed above are overcome in the present invention through the surprising finding that a decrease in release rate above a critical temperature, which can be very near to skin temperature, is possible by a novel arrangement of thermoresponsive polymer and platelet-shaped solid crystals. This reverse temperature dependence, which stands in sharp contrast against the almost universal increase in release rate with increased temperature in essentially all known materials, provides in this invention for dermal and transdermal release of active from materials, such as fabrics, that can be washed in warm or hot soapy water without undue loss of active.

In circumstances, including but not limited to medicament-releasing fabrics and bedding, where uniformity of release rate is relatively more important than temperatures response (e.g., where cold water wash is used), other embodiments disclosed herein over the shortcomings of the prior art through the attachment or inclusion of nested-polymer rod shaped particles in which solid active is dispersed, and wherein said particles obey certain mathematical relations between the geometric and kinetic parameters that result in a near-constant rate of release substantially independent of conditions external to the particles. In such embodiments, wasteful release of active is limited simply by the relatively small proportion of time spent in conditions of wasteful release, e.g., during washing operations, shelf-life prior to application, etc. In a novel solution to this challenge, particles are disclosed comprising solid dispersions of active compounds dispersed in nested or layered arrangements of at least two distinct polymers, wherein at least one of said polymers, polymer A, or the “outer polymer”, is substantially impermeable to the active compound, another of said polymers, polymer B, the “inner polymer”, is host to the dispersed active and is selected so as to yield dissolution-limited release of the active upon application, and in which the surface area of polymer B is substantially though not completely covered by polymer A, the percentage of area coverage being between 80% and 99.9%, more preferably between about 85% and 99.5%, and most preferably between about 90% and 99%, said particles being well suited for, e.g., attachment to fabric, such as an article of clothing or bedding, where they can serve to release active into the skin for local or systemic delivery, and yet substantially retain active during a laundry wash cycle. The particles are most preferably rod-shaped, coated with an impermeable skin except at one or both ends, confining release to a small area thus allowing for extended release over long time periods; the release rate is substantially unaffected by changes in the local environment of these particles, so that loss of active in the laundry is strongly limited by virtue of the limited time that the particle spends in the wash cycle as compared to in actual application, e.g., in contact with skin.

It is an object of this invention to provide hot-washable fabrics that are loaded with active, and which in a normal washing machine hot wash cycle do not lose more than about 25% of the active present just before the wash, more preferably do not lose more than about 12%, and most preferably do not lose more than about 7% of the active.

It is a further object of this invention to provide active-releasing materials from which the decrease in release rate of the active in water when changing from Tc−5° to Tc+5° is at least 10%, more preferably at least 50%, and most preferably at least 75%.

It is another object of this invention to provide multilayered polymer-based compositions of matter that release at a near-constant rate over most of the duration of an extended release profile, the constancy of release being due to the dissolution-limited nature of the release mechanism, and the extended lifetime of release being enabled by the restriction of the non-occluded area over which release can occur from the inner, active-loaded polymer layer. The percentage of occluded area restricting release of active from the active-loaded inner polymer is between 80% and 99.9%, more preferably between about 85% and 99.5%, and most preferably between about 90% and 99%.

It is a further object of this invention provide sustained-release compositions that provide not only for near-constant drug release, but also for independent control of release rate and duration of release through simple adjustment of the dimensions of the releasing material.

It is a further object of this invention to provide multilayered polymer-based rod-shaped particles that release at a near-constant rate over most of the duration of an extended release profile, the constancy of release arising from the dissolution-limited nature of the release mechanism, which in turn results from conformance to the following mathematical conditions:
the ratio D/(K·u) is greater than 1, more preferably greater than 10, and most preferably greater than 100;
the ratio 1K/D is less than 0.1, and more preferably less than 0.06;
the aspect ratio 1/d is preferably between 1 and 50, more preferably between 2 and 20, and most preferably between 2 and 10.

where 1 is the length of the rod-shaped particle, d is the diameter of the rod, D is the diffusion rate and K the dissolution constant of the active in the interior of the particle, and u=1 cm is a standard unit of length.

It is a further object of this invention to provide compositions comprising area-restricted dissolution-limited release particles attachment to fabric, such as an article of clothing or bedding, where they can serve to release active into the skin for local or systemic delivery, and yet substantially retain active during a laundry wash cycle.

### DESCRIPTION OF THE DRAWINGS

FIG. 1A is a schematic illustration of a crosslinked hydrogel embodiment of the present invention. An exemplary diffusional path of active out of the hydrogel is indicated by element 20.

FIG. 1B illustrates a crosslinked hydrogel of the invention above the critical collapse temperature Tc. The large increase in polymer concentration and the resulting “log-jam” formed by the platelets is illustrated. An exemplary, and highly tortuous, diffusional path of active out of the hydrogel is indicated by element 30.

FIG. 2 illustrates an embodiment of the invention in which the active is in the form of micro/nanocrystals (indicated by hexagon-shaped symbols) situated inside liquid or liquid crystalline domains 51.

FIG. 3 shows a photograph showing the results of a color-indicator test comparing the release of a colored marker, merocyanine 540, from two identical hydrogels of the invention that were held in water at different temperatures for 60 minutes. The water in which the hydrogels were soaked was removed and treated with a reducing agent to reveal the amount of dye that was released from each hydrogel during the 60 minute release period. A stronger color indicates more dye was released. The color strength in the cold-water sample, labeled “C”, is strikingly more intense than the color in the hot-water sample, labeled “H”. This demonstrates that the release rate of dye from the hydrogel of this invention is much higher at 19 degrees C. than at elevated temperature (approx. 45 degrees C.).

### DETAILED DESCRIPTION

The present document discloses novel and specific arrangements of crosslinked poly-N-isopropylacrylamide hydrogels comprising thin, diffusion-blocking solid particles of (usually platelet-shaped crystals), and also comprising active-containing particles or droplets for release into mammalian tissue, as well as precursor compositions that convert or can be converted to such arrangements, e.g., via polymerizing or crosslinking the precursor. The invention is based on the novel insight that such arrangements properly configured are able to defy the normal and persistent increase of release—whether diffusion-limited, dissolution-limited, or other—with increase in temperature. As illustrated schematically in FIG. 1A, at temperatures below a critical “collapse” temperature, the poly-N-isopropylacrylamide (PNIPAM) based hydrogel is in a relatively swollen, or expanded, configuration. Above this temperature it converts to a “collapsed”, or relatively un-swollen, tight configuration, as illustrated in FIG. 1B. Element 10 in FIG. 1A is a poly-N-isopropylacrylamide chain, 11 is a crosslink point in the crosslinked PNIPAM network, 12 is an active-containing particle, 13 is the location of a molecule of active inside a particle, 14 is the location of a molecule of active in the aqueous continuum of the hydrogel, 15 is a platelet-shaped crystal, and 16 is a point lying outside of the hydrogel; 17 is a point lying on the hydrogel surface, the interface between the hydrogel whatever lies exterior to the hydrogel. Preferably the collapse or de-swelling when the critical temperature is crossed is relatively fast, and results in the rapid formation of a diffusion-restricting “skin” at the surface of the hydrogel. In the preferred case where platelet-shaped crystals are embedded in the hydrogel, the hydrogel collapse induces a pronounced crowding of the platelets and an effective “walling off” of diffusion, such that the average diffusion time of a molecule of active across the hydrogel increases substantially over that in the swollen state, in contradiction to the decrease in average diffusion time that would normally be associated with a shrinking of hydrogel thickness. Illustrating this is element 20 in FIG. 1A and element 30 in FIG. 1B, each indicating a representative diffusion path taken by an active molecule in the act of escaping the diffusion-restricted hydrogel. Due to the more tortuous path required, the total diffusion distance can be much longer in the de-swollen case (FIG. 1B) that in the swollen case even though the end-to-end distance is shorter due to the reduced thickness of the de-swollen gel. For most applications, when the temperature subsequently drops to a temperature below the critical temperature, the reversion back to the more swollen state is preferably slower than the prior conversion to the de-swollen state.

FIG. 2 shows a preferred arrangement in which the active is in the form of solid particles, usually micro- or nano-crystals, indicated in the figure by element 50, which may be immersed in a liquid (or liquid crystalline) medium 51. The liquid droplet 51 may in turn be dispersed in a particle 52 (for example, the liquid of 51 could be water, inside of an oil droplet 52, in an approach that utilizes a W/O/W double emulsion), or the particle 52 may consist essentially of the liquid droplet 51 (that is, basically 51=52), in for example an approach that utilizes an O/W single emulsion. As in FIG. 1, the particle 52 is embedded in a platelet-containing PNIPAM hydrogel 53. In such an embodiment of the invention, release of the active from the hydrogel requires dissolution of the active micro/nanocrystal, and when the conditions described herein for dissolution-limited release are followed, a substantially constant release rate will result. It is also permissible that solid particles of active 50 are embedded directly in the hydrogel with no intervening liquids 51 or 52; in such an arrangement, the term “microparticle” as used herein refers to a solid particle of active, which is either an amorphous solid particle or a micro- or nano-crystal of the drug or active.

Critically, the reverse temperature dependence of this novel composition and arrangement, which yields a substantially lower rate of active release above the critical temperature, is particularly useful for providing active-releasing materials that can be exposed to certain elevated temperature environments without excessive loss of active; a normal wash cycle, such as with a household washing machine in normal operation with about a 35-minute start-to-finish run time, should result in the loss of not more than about 25% of the active that was in the material before washing, more preferably not more than about 12%, and most preferably not more than about 7%. Besides a washing machine warm/hot cycle, such elevated temperature environments also include, but are not limited to, those encountered in: steam cleaning, dry cleaning, ironing, blow-drying, bathing, showering, contact lens washing/sterilization, curing or drying a cast, applying a poultice, cauterizing a wound, and diathermy treatment. As the temperature is increased from 5 degrees Celsius below Tc to 5 degrees above Tc, the release rate of active from the hydrogel into water under infinite-sink conditions decreases by preferably at least 10%, more preferably by at least 50%, and most preferably by at least 75%. For embodiments of the invention that are provided as liquid, crosslinkable precursors as discussed at length herein, then upon completion of formation of the final crosslinked, platelet-loaded hydrogel according to the methods and spirit of the invention as described herein (in particular, where sedimenting-out of the platelets before crosslinking is substantially avoided, by timely processing), the preferred decrease in release rate when changing from Tc−5° to Tc+5° is at least 10%, more preferably at least 50%, and most preferably at least 75%.

Release of active into mammalian tissue, most preferably though not exclusively human skin, is the central purpose of the embodiments of this invention. For a material to be suitable as a release vehicle, it must satisfy a number of criteria: it should be of relatively low toxicity; it should exhibit low allergenic potential and low skin irritation; and most importantly it must release the active at a rate that delivers an efficacious and reasonably safe dose in the time anticipated for the vehicle-tissue contact. Each of these criteria should be taken in light of the seriousness of the condition/disease, and of the normal operational parameters of medicine and pharmaceutics.

It should be evident to one skilled in the art that the active being released is not water. Indeed, the collapse of the hydrogel with the expulsion of water is what happens in the hot wash, not at the application temperature. Furthermore, it is known in the field of dermatology that while hydration of the skin is a common therapeutic goal, this generally cannot accomplished by simply releasing water onto the skin.

Incorporation/Dispersing of Active.

In the course of practicing this invention, it is necessary to obtain or produce an aqueous dispersion of the active ingredient(s), such that particles or droplets comprising the active are dispersed in an aqueous continuous medium. If the active is a liquid at ambient temperature, as in the case of where the active is an essential oil, for example, then it is not necessary to solubilize this active, as it can be dispersed directly if desired in the form of dispersed liquid droplets. Nevertheless there could be reason for diluting this liquid active with another liquid, for example a triglyceride; this could be for purposes such as reducing the release rate, reducing stinging or tissue irritation, reducing the amount of N-isopropylacrylamide in the oil phase, improving the stability of the dispersion, etc.

If the active is a solid at ambient temperature, then there are two general approaches to dispersing the active. In one approach the active is solubilized in a liquid, and the liquid containing dissolved active is then dispersed in the form of droplets. In the context of this invention, the liquid-droplet nature of the dispersed active-containing fluid remains fundamental whether the droplets are in the form of, e.g., emulsion droplets, liposomes, or the interior of a microcapsule.

Alternatively, a solid active can be dispersed in the form of solid particles. Solid active particles or crystals can be: A) dispersed in a water-insoluble liquid, which is in turn dispersed in water as an O/W emulsion; B) dispersed in a water-insoluble or water-miscible liquid that forms the interior (or “core”) of microcapsules that are dispersed in water; C) dispersed in a polar solvent such as water that forms the interior phase of a liposomal dispersion; or D) dispersed in water directly. (In terms of the terminology used herein, in case D the term “microparticle” as used herein refers to the solid particles of active). The choice determines, among other things, the local milieu in which the active dissolves, which has a strong effect on the dissolution kinetics and potentially on the release kinetics and profile.

For many reasons as discussed in this disclosure, the use of essential oils, plant oleoresins, and other plant natural extracts is of high utility in the practice of this invention. When used as active ingredients, essential oils, and plant extracts prepared using organic solvents, lend themselves well to O/W emulsification, and can form the core of water-dispersible microcapsules provided the oil does not significantly solubilize the shell material. Likewise, as inactive ingredients, essential oils are excellent solvents of crystalline actives, including though not limited to active pharmaceutical ingredients (APIs), and when containing dissolved active, can also be dispersed in O/W emulsion or microcapsule form. However, in addition to these possibilities, another approach exists in which active solid particles or crystals are dispersed in an essential oil or other water-insoluble plant extract, which is in turn emulsified in water; this can yield a dissolution rate that is drastically different from the dissolution rate of the same particles or crystals in water.

Solubilization.

In embodiments where a solid active is solubilized in a liquid, two fundamentally different cases exist: either the liquid is water-miscible, or the liquid is water-immiscible. When the solvent is water-miscible, then in the context of this invention, it is necessary to encapsulate this active-containing solvent, e.g., in a liposome or microcapsule, in order to benefit from the invention. It is neither compatible with, nor within the spirit of, this invention to simply solubilize the active ingredients directly within the aqueous domains of the PNIPAM hydrogel, because in such an arrangement, de-swelling of the PNIPAM hydrogel with increase in temperature would expel water and thus release large amounts of active, in opposition with the main object of this invention. In contrast, while an active is encapsulated within a gel-embedded liposome or microcapsule as prescribed in this disclosure, it is substantially protected against release through gel collapse alone, and is subject to the release-controlling mechanisms that are the basis of this invention. Indeed, it is preferred in the invention that the partitioning of active between particles/droplets and the hydrogel continuum is such that, at the beginning of active release, less than about 25% of active in the hydrogel is in the hydrogel continuum, indicated by 14 in FIG. 1, as opposed to the microparticle interior. More preferably the fraction of active in the hydrogel continuum at the beginning of active release is less than about 10%. As active is released and drained out of the microparticles, this “free” fraction may increase, and thus the ranges given above refer to the early stage of the release process, when only a small amount of active has been released from the hydrogel.

If a solid active is solubilized in a water-immiscible liquid, such as for example an essential oil, then there are many arrangements that can be invoked to control the release of active, as per the invention. The term “plant essential oils” is intended to include, in addition to those cited elsewhere herein, essential oils from the following:

allspice berry, amber essence, anise seed, arnica, balsam of Peru, basil, bay, bay leaf, bergamot, bois de rose (rosewood), cajeput, calendula (marigold pot), white camphor, caraway seed, cardamom, carrot seed, cedarwood, celery, German or Hungarian chamomile, Roman or English chamomile, cinnamon, citronella, clary sage, clovebud, coriander, cumin, cypress, eucalyptus, fennel, Siberian fir needle, frankincense (olibanum oil), garlic, rose geranium, ginger, grapefruit, hyssop, jasmine, jojoba, juniper berry, lavender, lemon, lemongrass, lime, marjoram, mugwort, mullein flower, myrrh gum, bigarade neroli, nutmeg, bitter orange, sweet orange, oregano palmarosa, patchouly, pennyroyal, black pepper, peppermint, petite grain, pine needle, poke root, rose absolute, rosehip seed, rosemary, sage, dalmation sage, sandalwood oil, sassafras, spearmint, spikenard, spruce (hemlock), tangerine, tea tree, thuja (cedar leaf), thyme, vanilla extract, vetivert, wintergreen, witch hazel (hamamelia) extract, ylang ylang (cananga) extract and components and mixtures thereof.

Of these essential oils, those which are labeled as GRAS (Generally Regarded As Safe) for certain modes of application are particularly preferred, and include: ylang ylang, clovebud, spearmint, ginger, patchouly, sandalwood, carrot seed, peppermint and mixtures of peppermint and thyme.

The following components of essential oils may also be particularly useful: methyl salicylate; ( [symbol] )-menthol; eugenol; isoeugenol; 2,6-dimethyl-2,4,6-octatriene; 4-propenylanisole; benzyl-3-phenylpropenoic acid; 1,7,7-trimethylbicyclo[2.2.1]heptan-2-ol; 2,2-dimethyl-3-methylenebicyclo[2.2.1]heptane; 1,7,7-trimethylbicyclo[2.2.1]heptane; trans-8-methyl-n-vanillyl-6-nonenamide; 2,2,5-trimethylbicyclo[4.1.0]hept-5-ene; 5-isopropyl-2-methylphenol; p-mentha-6,8-dien-2-ol; p-mentha-6,8-dien-2-one; beta-caryophyllene; 3-phenylpropenaldehyde; mixed geranial and neral; 3,7-dimethyl-6-octenal; 3,7-dimethyl-6-octen-1-ol; 4-allylanisole; ethyl 3-phenylpropenoic acid; 3-ethoxy-4-hydroxybenzaldehyde; 1,8-cineole; 4-allyl-2-methoxyphenol; 3,7,11-trimethyl-2,6,10-dodecatrien-1-ol; 1,3,3-trimethylbicyclo[2.2.1]heptan-2-ol; 1,3,3-trimethylbicyclo[2.2.1]heptan-2-one; trans-3,7-dimethyl-2,6-octadien-1-ol; trans-3,7-dimethyl-2,6-octadien-1-yl acetate; 3-methyl-2-(2-pentenyl)-2-cyclopenten-1-one; p-mentha-1,8-diene; 3,7-dimethyl-1,6-octadien-3-ol; 3,7-dimethyl-1,6-octadien-3-yl acetate; p-menthan-3-ol; p-menthan-3-one; methyl 2-aminobenzoate; methyl-3-oxo-2-(2-pentenyl)-cyclopentane acetate; methyl 2-hydroxybenzoate; 7-methyl-3-methylene-1,6-octadiene; cis-3,7-dimethyl-2,6-octadien-1-ol; 2,6,6-trimethylbicyclo[3.1.1]hept-2-ene; 6,6-dimethyl-2-methylenebicyclo[3.1.1]heptane; p-menth-4(8)-en-3-one; p-menth-1-en-4-ol; p-mentha-1,3-diene; p-menth-1-en-8-ol; 2 isopropyl-5-methylphenol.

These essential oils and essential oil components constitute a very powerful array of solubilizing liquids, in the context of this invention. Due to the varied physicochemical properties of these compounds, for a given active, a good solvent for that active could be found within the essential oils above, but in addition, it may well be possible to find a poorer solvent that yields a low dissolution rate constant.

Microcapsules.

Embedding active-containing microcapsules in a crosslinked PNIPAM hydrogel of the invention can follow a range of designs. The release kinetics can be diffusion limited—including the possibility where diffusion through the microcapsule shell is the rate-determining step—or shell-dissolution limited, or active-dissolution limited if the active is insoluble in the microcapsule core and dispersed in the core as micro/nanocrystals.

Many processes and compositions for core-shell microcapsules are known in the art, with processes including but not limited to coacervation, complex coacervation, admicellization, interfacial polymerization, emulsion polymerization, spray-drying, fluid-bed coating, sol-gel, co-extrusion, antisolvent-based methods, heterocoagulation, and layer-by-layer methods. Since the microcapsules can be formed prior to addition (and polymerization) of the other matrix components such as NIPAM and platelets, most of these microcapsule processes and compositions can be used, provided: A) the shell is of relatively low solubility in water; and B) the toxicity of the microcapsule components is low enough to be acceptable in the given application. A water-soluble polymer can only be used as the shell material if it is heavily crosslinked (thereby rendering it insoluble), and this can be a particularly good approach if the shell material comprises crosslinked PNIPAM. If a water-insoluble polymer is used, crosslinking is in general not required, unless the polymer is soluble in the core material—which is a very real possibility in the case where essential oils make up a significant portion of the core. Preferably the solubility of the shell in water is less than about 10 mg/mL, and more preferably less than about 2 mg/mL. Also, preferably the solubility of the shell in the core is less than about 50 mg/mL, and more preferably less than about 10 mg/mL. Preferred shell materials are polylactide, poly(glycolide-co-lactide) (or PLGA), xanthan gum, gum tragacanth, chitosan, polyurethane, polyurea, polyamide, polyacrylate, polystyrene, polybutadiene, crospovidone, silicone, starches, fructans (such as inulin), and cellulose and its derivatives; also preferred are graft copolymers that result from interfacial polymerizations such as that described in U.S. Pat. No. 7,736,695 to Schwantes. In addition to polymers, other useful shell materials include insoluble minerals, e.g., calcium phosphates, hydrophobic lipids such as solid fats, water-insoluble amino acids and multivalent salts thereof, inclusion complexes, and coagulates of nanoparticles.

One skilled in the art will recognize several of these preferred polymers as biodegradable polymers, such as PLGA, which break down chemically over time when in contact with body fluids. This is one mechanism by which the microcapsules can release active. Two other mechanisms for release of microcapsules that are compatible with the present invention are pressure, and shear. Such mechanical forces could be invoked to initiate the release of active, for example, when the head hits a pillow, or when a wound dressing is applied and rubbed or pressed to prime the release.

While the use of microcapsules in prior art applications generally requires that the microcapsules break open in order to release active, in the present invention this is not necessary, since slow diffusion of molecules of active through the intact shell is an acceptable means of release. Indeed, if the characteristic rate of diffusion across the shell thickness is slow compared to the rates of active dissolution (if required at all) and of diffusion through the hydrogel, then the overall rate of active release is shell-diffusion limited. Interestingly, if the shell is undergoing a slow degradation (as with PLGA, for example), then this could have the effect of accelerating the overall release rate over time, which could in part, or in full, compensate for the normal decrease in release rate associated with diffusion-limited release, thus producing a more constant release rate.

Liposomes.

Liposomes by definition have an aqueous, or more broadly a polar solvent-rich, core. They are therefore fundamentally distinguished from oil-core microcapsules, and yet may still be employed in the present invention. Two distinct forms of liposomes are possible: one in which the shell, i.e., the lipid bilayer, is in the crystalline state, and the other in which it is in the fluid state. More precisely, there is a fluid bilayer to crystalline bilayer transition temperature, and this can be either above, or below, the application temperature, corresponding to the crystalline and fluid bilayer cases, respectively. If the bilayer is in a crystalline state, then diffusion of the active across the bilayer is extremely slow, generally too slow to be considered in an active-releasing state.

If the bilayer defining the liposome is in the fluid state, then liposome-encapsulated actives can diffuse across the bilayer from the core and exit the liposome. Generally, at similar molecular weights, the more hydrophobic a molecule is the faster it will diffuse in the bilayer. And since the more hydrophobic a molecule is the slower it dissolves in the aqueous core of the liposome, then this contrast allows for relatively simple control of the ratio of dissolution kinetics to diffusion kinetics, for a solid active encapsulated in the liposome. Simply put, release of a more hydrophobic molecule will be more dissolution limited, whereas a more hydrophilic molecule will be more diffusion limited. As discussed in much more quantitative detail herein, a dissolution limited release will tend to lead to a more linear, constant-rate, release profile, whereas a diffusion limited release will tend to lead to a less linear release profile, which is less favorable for the user.

O/W Emulsions.

Oil-in-water emulsions provide a very versatile starting point for compositions of the present invention. As in other microstructures discussed herein, the active can either be solubilized in the oily core or dispersed in the oily core in the form of micro/nanocrystals. But what makes this approach so versatile is the large range of solvent properties available in the emulsion interior phase. At one extreme are very poor solvents such as long-chain triglycerides (fats). LCTs are extremely hydrophobic, and of rather high MW as solvents go (roughly 800 MW), which in turn makes their viscosities one or two orders of magnitude higher than those of low MW solvents such as water and essential oils. (E.g., the viscosity of triolein at 20° C. is approximately 86 cSt). Properties like this make it possible to achieve a low dissolution rate constant even with a hydrophobic active, and using the methods of this invention a low dissolution rate can be used to achieve a linear or near-linear release profile. Furthermore, triglyceride-based O/W emulsions come in compositions that are even approved for direct intravenous administration, including formulations such as Intralipid that are infused at hundreds of milliliters per day. At the other extreme are essential oils that are rich in low-MW aromatic and aliphatic hydrocarbons, and punctuated with oxygen-containing compounds such as phenolic and hydroxylated compounds. Powerful, amphiphilic solvents such as this are needed for difficulty-soluble compounds, and for a given active, may even be required just to achieve an active-dissolution rate high enough to be practical for a particular application.

W/O/W Multiple Emulsions.

One skilled in the art will recognize that there are many advantages inherent in having active disposed in an aqueous medium. One very important advantage is the very practical one that nearly every drug or serious candidate for a drug has been studied from the point of view of solubility in water, and in many cases information that is at least predictive of dissolution rate. Other advantages follow from the pure hydrophilicity play that results from the choice of water as the solvent, and the flexibility of the aqueous milieu afforded by the 0-14 pH range.

There is a clear difference between disposing an active compound directly in a hydrogel, and disposing it in the interior water phase of a W/O/W multiple emulsion. In the latter case, a molecule of active must traverse an oil domain, and two interfaces, in order to be released. If the emulsion interface has substantial viscoelasticity, as in the case of Pickering emulsions but also in typical emulsions wherein phospholipids are used as the main emulsifier, then this can strongly reduce diffusion and release rates. Embedding of the emulsion droplet in a hydrogel means that long-range diffusion of active cannot occur by droplet movement alone, and if the active is dispersed in solid form, release requires dissolution in an aqueous droplet. In the case of a less hydrophilic active, one that can nevertheless be coaxed into partitioning into the dispersed (interior) water phase, dissolution in water might well be slow enough to result in dissolution-limited release, and thus a linear release profile. Coaxing a crystal into an aqueous domain can in many cases be accomplished by one or more of the following: adjusting the pH of the aqueous droplets in accordance with titrating groups on the active; mixing water with a polar co-solvent such as glycerol, a formamide or acetamide, etc.; reducing the “attractiveness” of the oil phase by choosing a poor solvent, such as a long-chain triglyceride or squalene; choosing a surfactant or dispersant that is effective at dispersing the active crystals in water but not in oil, etc.

Liquid Crystalline Particles.

A series of patents by Anderson (U.S. Pat. Nos. 6,638,621 and 7,713,440 and the other members of these families), and one by Landh and Larsson (U.S. Pat. No. 5,531,925) have provided highly detailed methods, compositions, and application test results for aqueous dispersions of lyotropic liquid crystalline particles loaded with active compounds. Most preferred in the context of the present invention are those in which the core of the particles (or, in the case of U.S. Pat. No. 7,713,440 the entire particle) consists of a “reversed hexagonal” or “reversed cubic” phase, or a mixture thereof. This liquid crystalline, active-containing core can be coated, or uncoated, as follows:
Uncoated, as in U.S. Pat. No. 7,713,440 to Anderson;
Solid-coated, as in U.S. Pat. No. 6,638,621 to Anderson; or
Coated with a liquid or lamellar liquid crystalline phase coating, as in U.S. Pat. No. 5,531,925.

While each of these particle formats can be used in the present invention, the most preferred is in most cases the first of these, namely the uncoated particles of U.S. Pat. No. 7,713,440 that are stabilized in dispersion without a coating phase at the surface of the particle. The most important exception to this general rule is when the active is substantially hydrophilic, such that it does not partition strongly enough into the particles without the solid coating, in which case the particle format of U.S. Pat. No. 6,638,621 is most preferred; this is the case when the partition coefficient measured between the liquid crystalline phase and water is less than about 10. But when this partition coefficient is greater than about 10, uncoated particles as per U.S. Pat. No. 7,713,440 are most preferred, and it should be noted that the dispersion stability requirements of central importance in U.S. Pat. No. 7,713,440 are lessened in the current invention due to the particle-immobilizing effect of the PNIPAM-based polymer. That is, it is really only necessary for the dispersion to be stable (against, e.g., creaming, flocculation, coalescence, sedimentation) long enough to perform the hydrogel-forming polymerization of N-isopropylacrylamide (and other comonomers and crosslinkers, etc.). This may be very short, only minutes in most cases; during that time there should preferably not be any strong shearing or fluid dynamics (such as might otherwise be useful in maintaining proper dispersion of the particles), since this will interfere with the setting up of a particle-immobilizing hydrogel, but on the other hand more “static” methods such as sonication are applicable.

As discussed at length in U.S. Patent Application No. 2002/0102280 to Anderson, as well as in U.S. Pat. No. 6,991,809 to Anderson, reversed liquid crystals produced from combinations of surfactants—particularly phospholipids—and certain powerful solubilizers of relatively low toxicity, such as essential oils and liquid vitamins such as vitamin E (tocopherol), provide powerful solubilization matrices for actives including pharmaceutical actives, and can be dispersed according to methods a) through c) above. Furthermore, in addition to solubilizing difficulty-soluble actives, the afore-mentioned essential oils, plant extracts, and tocopherols in the patents of Anderson can themselves be highly effective and safe actives, as discussed in considerable detail herein. Especially preferred solubilizers of solid (crystalline) actives are tocopherols (particularly alpha-tocopherol), tocols, castor oil, and the essential oils of ylang ylang, clovebud, cedarwood, spearmint, ginger, patchouli, santalwood, carrot seed, fir needle, rosemary, jasmine, fennel, palmarosa, oil of bay, peppermint and mixtures of peppermint and thyme.

In preferred embodiments, effective solubilizers, such as those listed above, are formulated together with water and with surfactants or lipids, which satisfy the definition of a surfactant known to one skilled in the art: namely, they form self-association structures such as micelles and liquid crystals when combined with water, and substantially lower the surface tension of water at low concentrations. Especially preferred surfactants and lipids are lecithin, phosphatidylcholine-rich purified lecithin, salts of docusate (di-ethylhexylsulfosuccinate) and closely related sulfosuccinates, poloxamers with HLB less than 10, sorbitan fatty acid esters particularly di- and tri-oleates, unsaturated monoglycerides, and ethoxylated castor oil fatty acid esters.

In the context of the present invention, one feature of lyotropic liquid crystalline particles that is particularly useful for achieving the goals of the invention is the high viscosity of reversed hexagonal and reversed cubic phase liquid crystals. The zero-shear viscosity of a typical phospholipid-oil-water cubic phase of the invention was measured to be in the range of billions of centipoise (and thus billions of times more viscous than water). These high viscosities can be utilized to yield low dissolution rates of solid active crystals dispersed in these liquid crystalline materials, thus enabling dissolution-limited release that is used in this invention to achieve a highly desirable constant or near-constant rate of release of active.

Micro/Nanocrystalline Active.

One of the most effective approaches in the current invention is to disperse the active in the form of microcrystals or nanocrystals, with the latter generally agreed to be crystals with less than 400 nm (0.4 micron) characteristic dimension. These micro/nanocrystals are dispersed in the particles or droplets that are, in turn, immobilized in the PNIPAM-based hydrogel of the invention. In most cases, the ultra-microscopic size of these crystals is driven primarily by the desire to confine each crystal within a single particle (or droplet, in the case of an emulsion), rather than by an intrinsic need for microscopic crystals.

The micro/nanocrystalline active is not to be confused with the inactive, platelet-shaped crystals that are discussed elsewhere herein. Indeed, it is not within the spirit of this invention for the main active or drug crystals to serve as the platelet-shaped, diffusion-limiting crystals required in the invention. While the platelet-shaped crystals may have some secondary activity, they are not the primary drug or active. One skilled in the art will recognize the concept of a “functional excipient”, which may be a fair description of the platelets in some embodiments. Alternatively, the platelets may have enough solubility to be released into the skin/tissue and have a desirable physiological or “drug” effect, but in general the platelet material is chosen to have very low solubility and/or dissolution rate.

Methods for producing small crystals of an active compound can be categorized according to whether larger starting materials are milled down to smaller size (the “top-down” approach), or microscopic crystals are engineered from the start (the “bottom-up” approach). Methods for milling include high-shear homogenization, high-pressure homogenization (also known as microfluidization), ultrasonication, wet milling, ball milling, and others. “Bottom-up” methods generally rely on precipitation or crystallization in the presence of size-reductive methods such as homogenization and sonication; alternatively, actives can be crystallized within microstructures, such as emulsion droplets, liposomes, microparticles, etc., that inherently limit the size of the resulting crystals.

In preferred embodiments, these microscopic crystals of active are dispersed within particles or droplets that play a central role in controlling the dissolution rate of the active crystals. Somewhat less preferred is the case where the crystals of active are dispersed directly in the hydrogel; this is less preferred because although it is simpler to execute, there is far less opportunity to adjust the dissolution rate of the crystals over a wide range, as may be needed in order to obtain the desired delivery profile.

Immobilization in Hydrogel.

An essential element in the end use of this invention is the “immobilization” of the active-containing particles in a crosslinked PNIPAM-based hydrogel. By “immobilization” is meant that the diffusion of particles within the hydrogel is strongly retarded by the polymer network, such that the ratio of the effective particle diffusion coefficient in the hydrogel to that in pure water is less than 1:10, more preferably less than 1:100, and most preferably less than 1:1000. Thousand-fold reduction in diffusivity is most preferably achieved, in the end-use product from this invention, by a high crosslink density, such that the average distance between crosslinks is significantly less than the average particle diameter. Intuitively, this means that the particles are entrapped in a web, or mesh, of three dimensional extent that is tight enough to retain the particles within a fixed portion of the mesh. Of course, this means that if the hydrogel moves, shrinks or expands, rotates, etc., then so will the particles entrapped within. But this is not evidence of diffusion within the hydrogel. Indeed, the simultaneous movement of particles in concert with motions of the hydrogel demonstrates the confinement of each particle within the hydrogel.

In addition to poly-N-isopropylacrylamide and NIPAM-rich copolymers discussed herein, a few other polymers have been reported that exhibit sharp de-swelling transitions above a critical temperature near body or skin temperature. Two such polymers, which can be used in the present invention, are both based on glycidyl methyl ether (GME) as the main monomer. One of these uses ethyl glycidyl ether (EGE) as a comonomer, introduced in a statistically random manner, to yield poly[(GME)-stat-co-(EGE)]. The second uses ethoxy ethyl glycidyl ether (EEGE) as the comonomer, which after copolymerization and acidic treatment must be further converted to the corresponding glycidyl N-isopropyl carbamate (GNIPC) repeating unit by reaction with N-isopropyl isocyanate. [See Weinhart et al. (2011) Chem. Comm. 47:1553].

Although, as stated above, a crosslinked hydrogel is essential for the end use of products from the invention, the invention can be in the form of a precursor to this hydrogel, containing NIPAM or PNIPAM, crosslinker, and optionally one or more polymerization catalysts. This can be provided in the form of a liquid, which can be conveniently sprayed, atomized, painted, spin-coated, etc., and simultaneously or subsequently polymerized. Thus, important embodiments of this invention comprise liquid formulations containing active-loaded microparticles, platelet-shaped crystals, and NIPAM or PNIPAM with crosslinker and optionally one or more polymerization or crosslinking catalysts, such that addition and/or activation of the catalyst(s) yields a crosslinked hydrogel comprising immobilized microparticles and platelets. At the time of polymerization and/or crosslinking, the microparticles and platelets are dispersed in the aqueous medium. In this disclosure, to avoid confusion, the term “dispersed” is reserved for liquid media and not hydrogels, and the term “immobilized” is used instead in the case of crosslinked hydrogels—despite the fact that the microparticles and platelets would be considered by one skilled in the art to be dispersed in the hydrogel after crosslinking, in accordance with common use of the term “dispersed”.

Several general approaches can be used, and optionally combined, to convert a liquid product (non-crosslinked precursor) to a crosslinked hydrogel:
Monomeric N-isopropylacrylamide is solubilized together with crosslinking monomer in water, and a radical initiator (e.g., a peroxide, persulfate, azo compound, etc.) is added; if the initiator is added to the solution “in the factory”, i.e., before reaching the end user, then this is a “one-pot” formulation; if the initiator is provided separately and then mixed into the monomer solution by the end user, then this can be referred to as a “two-pot” system.
N-isopropylacrylamide is polymerized at the factory in the presence of functionalized chain-transfer agents or terminators, functionalized so that reactive groups are present at both ends of the relatively short polymeric or oligomeric chains; other “linker” compounds co-solubilized in water along with these chains contain a number of reactive groups that can be activated to crosslink the short chains together into crosslinked networks, with each linker molecule preferably able to form a covalent bond with 3 or more chain end groups; alternatively, the chain-transfer or terminator molecules can be such that they yield 2 or more reactive groups on one or both ends of a significant fraction of the short chains, preferably at least 5% of these chain ends, and more preferably at least 20% of these chain ends. The reaction that links the reactive end groups of the short chains, in some cases with linker compounds, may be triggered by the end user either through external means, such as heat or UV light, or by the addition of one or more catalysts. Preferably, the reactive groups (call them “type X”) at the chain ends do not react with each other readily, and neither do the reactive groups (call them “type Y”) on the linker molecules, but type X groups react readily with type Y groups, at least in the presence of catalyst; such an arrangement limits non-productive reactions during storage and shipping prior to use, when linker and chains are mixed.
N-isopropylacrylamide is polymerized at the factory in the presence of co-monomers that retain reactivity after the polymerization; in contrast with the previous approach, the chain length (MW) of these functionalized chains can be relatively short, very long, or anything in between; however the average distance between functional groups in the polymer should be about equal to or less than the average microparticle size. The final crosslinking—which, as in both previous approaches, can either be performed at the factory or by the end user—is accomplished by either reacting the functional groups in the chains with each other, or with linker molecules capable or forming covalent bonds with at least 2 functionalized groups in the chains.
N-isopropylacrylamide in aqueous solution is polymerized at the factory in the presence of the full load of crosslinker, so as to create a fully crosslinked hydrogel at the factory. Thus, in this approach, there is no option for the end user to perform the crosslinking. However, if the hydrogel is produced in the form of beads, or particles of any shape, and these are dispersed in water, then this dispersion may be applied by the end user using many of the methods that apply to liquids. Thus, a particularly versatile and easy to use embodiment of the invention is an aqueous dispersion of hydrogel beads, said beads comprising active-loaded microparticles, platelet-shaped solid particles, and crosslinked PNIPAM; the average size of a hydrogel bead in the swollen (i.e., non-collapsed) state lies preferably between about 1 micron and 1 centimeter, more preferably between about 10 microns and 3 millimeters, and most preferably between about 30 microns and 1 millimeter.

Whether the polymerization of monomeric NIPAM to poly-NIPAM is carried out in the factory or by the end user, it will generally be necessary, or at least strongly preferred, to remove oxygen from the liquid, due to the inhibiting effect of oxygen on polymerization of NIPAM. Sparging with an inert gas, such as nitrogen or argon, should generally be used at the factory, so that even in the case where the end user performs the polymerization, oxygen is at least relatively sparse when the liquid-containing package is open. In addition, if a monomer-loaded liquid is sprayed or applied by other means that result in oxygen uptake into the precursor liquid, then it may be necessary to incorporate oxygen scavengers in the liquid (or in a separate add-to liquid). Preferred such scavengers are sodium sulfite, erythorbate, ascorbate (Vitamin C), micronized iron, hydrazine, carbohydrazide, methylethylketoxime, diethylhydroxylamine, and combinations of these with trace amounts of cobalt salts as catalysts. Sodium sulfite, ascorbate, and erythorbate are most preferred as they are relatively non-toxic and approved by regulatory agencies for food and drug applications. Oxygen scavengers that react with radicals such as those present during the NIPAM polymerization, such as quinones, are of course not favored as they can inhibit the polymerization.

In all of the above approaches, it may be advantageous to add a co-solvent to the water in order to improve the solubilities of monomers, crosslinkers, linkers, etc. Such co-solvents include ethanol, methanol, propanol, DMSO, DMF, dimethylacetamide, Nmethylacetamide, acetone, and glycerol. Especially preferred are volatile, water-miscible co-solvents with boiling points lower than that of water, so that they can be removed by evaporation. Alternatively, co-solvent can be removed simply by soaking the hydrogel in water.

Interpenetrating networks (IPNs) of crosslinked PNIPAM and another, separate crosslinked polymer, such as vinyl-terminated polyurethane, can be utilized in this invention in order to increase the physical toughness of the hydrogels.

As discussed below, in many embodiments it is advantageous to include, in addition to the components cited above, one or more components that provide for substantial adhesion of the hydrogel to a substrate material.

Reverse Temperature-Dependence.

This invention utilizes the shrinkage—“collapse”—of crosslinked PNIPAM hydrogels that occurs with an increase in temperature above a critical temperature, Tc, which lies near or slightly above the temperature of human skin. For simple PNIPAM hydrogels, with low levels of co-monomers and crosslinker, Tc is close to 33° C. When N-vinylpyrrolidone (NVP) is incorporated as a comonomer (with 1 hydroxycyclohexylphenylketone as UV-activated initiator), Tc can be increased, to, e.g., as high as 43° C. when the NVP concentration is 34%. Small concentrations of 2-(acrylamido)-2-methyl propane sulfonic acid (AMPS), or other ionic co-monomers, increase Tc substantially. Thus, using simple co-monomers, the collapse temperature can be accurately tuned to any desired temperature from 33° C. to at least 50° C., covering the range from human skin temperature to the temperature of hot water as found in the hot cycle of a washing machine. In addition, by using a hydrophobic co-monomer, such as butyl methacrylate, Tc can be decreased, for applications that call for it. Perhaps more importantly, for applications where it is desired to employ cationic or anionic co-monomers, e.g., for adhesion to a substrate, then hydrophobic co-monomers such as butyl methacrylate can be used to counteract the Tc-increasing effect of the ionic co-monomers.

Incorporation of Platelets.

Synergism between PNIPAM collapse and diffusion-blocking platelets, so as to substantially reduce the release rate as the temperature is raised above the collapse temperature Tc, is at the core of this invention. As pointed out above, this is in opposition to the prior art, which teaches that collapse of a drug-laden PNIPAM hydrogel increases the release rate. Thus, the platelet-shaped solid particles of the invention are central to all embodiments and completely change, even reverse, the relationship between temperature and release rate.

The platelets in this invention should possess the following properties: 1) a low, preferably extremely low, diffusivity of active across the platelet; 2) a large ratio between the width of the platelet face and the platelet thickness; and 3) in the case of a bead (particle) hydrogel format, a size that is smaller than about one-half the hydrogel bead size. If the platelet is described at least approximately by a parallelepiped with dimensions L×W×T, where L≧W>>T with T being the platelet thickness, then the “aspect” ratio W/T should preferably be greater than or equal to about 3, more preferably greater than or equal to about 5, and most preferably greater than or equal to about 10. If the aspect ratio were close to unity, then these more equant crystals would act to “bulk up” the hydrogel above the collapse temperature, instead of allowing it to shrink as desired in the invention. One skilled in the art will recognize that the ratio W/T was chosen instead of the (generally larger, by definition) L/T ratio because of the fact that elongate crystal habits (where L/T is much larger than W/T) are not well suited for the diffusion-blocking crystals of the invention.

In the case of a hydrogel film, wherein the film thickness is small compared to the extent in the other two dimensions, it is desired, though not essential, for the platelets to have a statistical bias toward lying roughly parallel to the film—that is, with the normal to the platelet face aligned approximately parallel to the film normal. It should be intuitively obvious to one skilled in the art that this maximizes the increase in the diffusion-blocking effect of the platelets as the hydrogel collapses, since the component of diffusion that leads to release is normal to the film surface. (I.e., diffusion normal to the hydrogel surface can rapidly lead to release, whereas diffusion parallel to this surface, or lateral diffusion, generally does not). If the film thickness is of the same order of magnitude as the platelet length L, or larger, then this alignment will occur naturally in most film deposition methods. If, on the other hand, the film thickness is large compared to L, say greater than or equal to about 5 L, then it will generally be necessary to perform the film deposition in such a manner that promotes alignment. Shear is the simplest way to achieve this: alignment throughout a relatively thick film can be promoted by moving the fluid during crosslinking with a motion that is parallel to the film surface, including but not limited to: a) a massaging, reciprocating motion; or b) a spreading of the film over a substrate. Examples of the latter are spin-coating, and blade-coating, both well known in the art. The crosslinking (and in some processes, the polymerization of NIPAM as well) must be well underway before the alignment of the platelets has had time to randomize, and this can be promoted by using one or more of the following: a rapid crosslinking reaction; increasing the viscosity by adding thickeners such as glycerol, polymer, or a gum such as xanthan or guar gum. A dilatant, or shear-thickening, additive such as corn starch can be even more effective in some deposition processes. A rheopectic additive is most preferred, as the time-course of shear in the deposition process can lead to a high viscosity that retains alignment. A rheopectic paste, such as a gypsum paste, can simultaneously provide the platelet-shaped crystals and the alignment-preserving rheopecty.

Platelet-shaped crystals occur in many crystalline materials, and are in fact on of the most common crystalline forms found, both in nature and in synthetic crystalline materials, and in both euhedral and subhedral crystals. Micaceous, foliated, rosette, lenticular, platy, smectite and lamellar are all names of crystal habits that are very well suited for the platelets of the invention. Clays, both pillared and non-pillared, are readily cleaved into platelet-shaped solids, and submicron clay particles, or nanoclays, are especially preferred in this invention. Phyllosilicates, including kaolinite, montmorillonite-smectice, illite, and chlorite phyllosilicates or clays, can quite generally be collected or broken down into platelet-shaped solids suited for the invention. More specifically, materials for making preferred platelet-shaped microcrystals to use in the compositions of the invention include montmorillonite, bentonite, mica, gypsum, kaolin/kaolinite, starch, cellulose derivatives, hydroxyapatite, graphite, attapulgite, halloysite, and hectorite. Most preferred are microcrystals derived from montmorillonite, mica, or gypsum. An especially preferred clay-derived platelet material is Optigel WX, an inexpensive material that according to Rockwood Additives Ltd is an “activated smectite product, [which] has a high swelling capacity in water and shows a marked thixotropic thickening effect.” It should be noted that platelets which swell in size in an aqueous milieu (such as the hydrogel of the present invention) increasingly upon increase in temperature may also be useful in the present invention to further decrease active release rates with increasing temperature.

In addition, crystals conforming to the crystal habits mammillary, plumose, radiating, and sphenoid may also be coaxed into thin, flat crystals that satisfy the requirements for the platelet-shaped crystals of the invention. This thin, flat morphology can be promoted by precipitation/crystallization of the crystals under shear or confinement conditions that promote platelet-shaped crystals, or by post-crystallization processes that result in the cleavage of mica-like sheets or platelets. The canonical example of the latter is, of course, the peeling of ultrathin mica layers from a piece of bulk mica. Examples of crystal habits that are very poorly suited for use as diffusion-blockers in the invention include acicular, botryoidal, columnar, coxcomb, cubic, dendritic, dodecahedral, equant, fibrous, filiform, hexagonal, Hopper, nodular, tuberose, octahedral, prismatic, pseudohexagonal, stalactitic, stellate, tabular, and wheat sheaf.

The methods listed above for the production of micron-sized or submicron-sized crystals can be applied to produce the platelets of the invention, and so will not be repeated here. However, in dealing specifically with crystalline materials that form thin platelets, and thus may well be in a layered structure of some form, exfoliating the crystals may be a relatively easy process compared to more typical micronization processes with non-lamellar crystals, and can provide ultrathin layers that are very easily micronized. For example, the lay person may be familiar with the ease with which mica is cleaved, or exfoliated, into extremely thin layers. One skilled in the art will recognize that materials such as clays can also be swollen and subsequently exfoliated with relative ease. Micronized cellulose is another example of a commonly-used material coming from a lamellar structure.

The platelets of the invention can be modified by covalent binding of organic groups, in order to, e.g., improve dispersibility and dispersion stability, promote compatibility with stabilizing surfactants and/or with microparticles, provide alternative mechanisms for alignment, provide mechanisms for control of platelet distance from the hydrogel surface, or allow covalent attachment of the platelets to the PNIPAM polymer network.

Active-containing microparticles must be co-formulated with the platelets, and this means that the stabilization methods for the microparticles and platelets must be compatible. For example, one could not use a cationic surfactant to disperse the platelets and an anionic surfactant to disperse the microparticles. One way to simplify this requirement is to stabilize the microparticles in dispersion by a surfactant of the same charge (positive or negative) as that of the platelet crystals, both evaluated at the intended pH of the final hydrogel, or, use nonionic stabilizers. One disadvantage of nonionic stabilizers is that many of the most commonly used nonionic stabilizers tend to lose effectiveness as the temperature is increased; however, it should be recognized that loss of dispersion stability after hydrogel crosslinking, viz., in the washing machine cycle, have far less effect than before crosslinking and may be a non-issue.

For platelets that have a net anionic charge at the application pH, the preferred microparticle-stabilizing surfactants are: docusate, dodecylsulfate, deoxycholic acid (and related cholates, such as glycocholate), tocopherol succinate, stearic acid and other 18-carbon fatty acids including oleic, linoleic, and linolenic acids, gentisic acid, hydrophobic amino acids including tryptophan, tyrosine, leucine, isoleucine, aspartic acid, cystine, and their N-methylated derivatives, particularly N-acetyltryptophan, myristyl gamma-picolinium chloride, phosphatidylserine, phosphatidylinositol, phosphatidylglycerol (particularly dimyristoyl phosphatidylglycerol), and other anionic and acidic phospholipids, ascorbyl palmitate, stearoyl lactylate, glycyrrhizin, monoglyceride citrate, stearyl citrate, sodium stearyl fumarate, JBR-99 rhamnolipid (and other biosurfactants from Jeneil Biosurfactant), cardiolipin, glycocholic acid, taurocholic acid, and taurochenodeoxycholic acid. Especially preferred anionic surfactants are: sodium oleate, sodium dodecyl sulfate, sodium diethylhexyl sulfosuccinate, sodium dimethylhexyl sulfosuccinate, sodium di-2-ethylacetate, sodium 2-ethylhexyl sulfate, sodium undecane-3-sulfate, sodium ethylphenylundecanoate, carboxylate soaps of the form ICₙ, where the chain length n is between 8 and 20 and I is a monovalent counterion such as sodium, potassium, ammonium, etc. The person with skill in the art will recognize docusate as the anionic moiety of the surfactant docusate sodium (also known as Aerosol OT), and dodecylsulfate as the anionic moiety of the surfactant sodium dodecylsulfate, or SDS. Surface-active polypeptides and proteins, such as casein and albumin, may also be used, although careful attention must be paid to the pH which will have an effect on the charge of the molecule.

For platelets that have a net cationic charge at the application pH, the preferred microparticle-stabilizing surfactants are: myristyl-gamma-picolinium chloride, benzalkonium chloride, alkyltrimethylammoniun surfactants such as cetyltrimethylammonium bromide, tocopheryl dimethylaminoacetate hydrochloride, Cytofectin gs, 1,2-dioleoyl-sn-glycero-3-trimethylammonium-propane, cholesterol linked to lysinamide or ornithinamide, dimethyldioctadecyl ammonium bromide, 1,2-dioleoyl-sn-3-ethylphosphocholine and other double-chained lipids with a cationic charge carried by a phosphorus or arsenic atom, trimethyl aminoethane carbamoyl cholesterol iodide, O,O′-ditetradecanoyl-N-(alpha-trimethyl ammonioacetyl) diethanolamine chloride (DC-6-14), N-[(1-(2,3-dioleyloxy)propyl)]-N—N—N-trimethylammonium chloride, N-methyl-4-(dioleyl)methylpyridinium chloride (“saint-2”), lipidic glycosides with amino alkyl pendent groups, 1,2-dimyristyloxypropyl-3-dimethylhydroxyethyl ammonium bromide, bis[2-(11-phenoxyundecanoate)ethyl]-dimethylammonium bromide, N-hexadecyl-N-10-[O-(4-acetoxy)-phenylundecanoate]ethyl-dimethylammonium bromide, 3-beta-[N—(N′,N′-dimethylaminoethane)-carbamoyl.

Nonionic surfactants can be used for stabilization, and can be effective whether the platelets are positively charged, negatively charged, or neutral. Examples of useful surfactants are long-chain hydrocarbons connected through various linkages to polyethylene glycol (PEG), sorbitol, oligosaccharide, or less preferably polyglycerol groups. Preferred nonionic surfactants include acetylated monoglycerides, poloxamers such as poloxamer 188 and Pluronic F-127, Tweens such as Tween 20, Tween 60 or Tween 80, Brij surfactants, ceteth-2, choleth, stearamidoethyl diethylamine, ammoniated glycyrrhizin, lanolin nonionic derivatives, DATEM, lauric myristic diethanolamide, methyl gluceth-120 dioleate, short-chain or unsaturated monoglyceride citrate, octoxynol-1, oleth-2, oleth-5, PEG vegetable oil, peglicol-5-oleate, pegoxol 7 stearate, poloxamer 331, polyglyceryl-10 tetralinoleate, polyoxyethylene fatty acid esters, polyoxyl castor oil, polyoxyl distearate, polyoxyl glyceryl stearate, polyoxyl lanolin, polyoxyl-8 stearate, polyoxyl 150 distearate, polyoxyl 2 stearate, polyoxyl 35 castor oil, tyloxapol, polyoxyl 8 stearate, polyoxyl 60 castor oil, polyoxyl 75 lanolin, polysorbate 85, sorbitan sesquioleate, sorbitan trioleate, stear-o-wet c, stear-o-wet m, stearamidoethyl diethylamine, steareth-2, steareth-10, trideceth 10, phosphatidylcholine, lysophosphatidylcholine, and tocopheryl PEG 1000 succinate.

In addition, non-surfactant compounds that are nevertheless dispersants can be extremely valuable in combination with surfactants, particularly in stabilizing microcrystals. Povidone (PVP, polyvinylpyrrolidone), gum Arabic, gelatin, pectin and modified pectins, and other natural gums and synthetic stimulants are particularly useful in this invention prior to crosslinking of the PNIPAM network. It should be clear to one skilled in the art that after the PNIPAM crosslinked network is fully formed, the importance of the surfactant stabilizers and dispersants is greatly diminished.

Release, Diffusion.

Most embodiments of the invention are geared toward releasing active, such as a drug or plant oil, and quite generally molecules of active will need to leave the active-loaded microparticles and diffuse their way through the hydrogel to the outer surface of the hydrogel. Upon reaching the hydrogel surface, a given molecule of active can be released in one of two ways: into the air by evaporation, or into the skin or other tissue. Generally the latter will be much faster, leading to the critically important conclusion that release of active is driven by contact with tissue, and losses due to evaporation are generally small compared to active released to the target tissue. Likewise, this feature minimizes excessive fouling of the air in the case of unpleasant or overpowering scents from actives such as natural oils.

Depending on the system and the application, it may or may not be important to maintain a high fraction of the unreleased (or “yet to be released”) active inside the microcapsules (represented by point 13 in FIG. 1), and a relatively small fraction in the aqueous domains of the hydrogel (represented by point 14), including in the non-collapsed state. This can be promoted by a combination of several features. First, the use of microparticles that contain strongly hydrophobic, amphiphilic, or surfactant excipients (inactives) can be used to maintain active by virtue of equilibrium partitioning. It is highly advantageous in such an approach that the volume ratio of hydrophobic, amphiphilic, and surfactant excipients in the microparticles to active compound is about 1:1 or greater. It should be noted that this is virtually guaranteed with the liquid crystal-based microparticles of Anderson reviewed above. And second, solvent conditions and/or active selection can be made to yield a very low solubility and/or diffusivity in the aqueous domains. As mentioned elsewhere herein, flexibility in aqueous conditions is somewhat limited by the need to maintain the collapse behavior of the PNIPAM-based hydrogel. Nevertheless the addition of co-solvents, particularly glycerol, adjustment of pH so as to yield a low fraction of ionically-charged active molecules, and perhaps most effectively, the addition of polymers or particularly natural gums that reduce active diffusivity by one or more of the following: ion-pairing with the charged fraction of active; increase of local microviscosity in the aqueous domains; formation of diffusion-blocking hydrocolloids; and hydrophobic interaction with the active. Using such methods can reduce the rate at which active reaches the hydrogel surface (exemplified by point 17 in FIG. 1) in the absence of skin contact, and at least approach a kinetic situation in which active is release from the microparticles only as it is taken up by tissue.

In contrast, in some applications it will be more desired to have active released at a substantial rate into the air. This may be the case where it is desired to deliver at least a significant fraction, or even majority, of active to the brain through the trigeminal neural pathway via the nose. One way to promote this is to produce a hydrogel film, or arrange hydrogel beads, in such a way that air-filled voids penetrate into the hydrogel region. For example, a hydrogel film/layer can be template by a spiked solid; the pre-crosslinking liquid precursor can be spread over a substrate and then covered with a solid having spiked protrusions, so that crosslinking in this configuration and removal of the spiked solid leaves behind air-filled holes in the hydrogel film. If these holes take up a significant fraction of the hydrogel outer region, then as active enters this region after diffusing out of microparticles, it can evaporate out of a hole even as the hydrogel surface away from the holes is in contact with the skin. Pronounced roughness of the hydrogel surface can provide for vapor paths that exit the hydrogel without touching the skin. Other geometries, or loose-fitting fabrics, can accomplish this as well. The effectiveness of such an approach can be further increased by reducing the diffusivity of the active in the aqueous domains of the hydrogel, using methods as discussed elsewhere herein, because earlier release out of the holes (or roughness, more generally) avoids longer diffusion paths out to the hydrogel outermost surface.

It should be understood herein that where “contact with skin” is discussed, this includes the case where one or more active-permeable layers of material intervene between the hydrogel and the skin (or other tissue) provided that the diffusivity of active across these layers is non-trivial, i.e., these layers are permeable enough to active that significant and therapeutic levels of active can be delivered. Such intervening layers are not depicted in FIG. 1, but are easily imagined.

It can also be effective in the practice of the invention to provide a removable cover over the hydrogel, which can be removed prior to use. Such a cover can protect against premature release of active during shipping and storage, or even between uses if the cover can be re-applied to the hydrogel surface.

Release Under Aqueous Saturation Conditions.

If the solubility of the active is of low solubility in the aqueous domains of the hydrogel, then it may be possible to achieve a constant or near-constant rate of delivery of active to the skin or target tissue. In this approach, the concentration of active in these aqueous domains remains approximately equal to the saturation concentration during most of the release process, and only as active is released from the hydrogel does more active dissolve or transfer into the hydrogel from the microparticles, to replace the “lost” (actually, delivered) active. In the case of uncoated microparticles, the rate of uptake of active into the skin is, in broad generality, low compared to the release from microparticles due to the much higher surface area of the totality of microparticles, all the way up to the point where nearly all the active is used up from the microparticles. Therefore the rate of release will be substantially controlled by the rate of uptake into the skin with the hydrogel at the active saturation concentration, and since this is independent of the concentration of remaining active in the microparticles, the release profile is expected to be approximately the same each day, or each evening for nighttime use. This does not mean that the release rate is constant throughout the evening, and in fact one should expect the release rate to diminish significantly throughout the night as the active concentration in the skin builds up, reducing the diffusion-driving concentration differential between the hydrogel and skin. However, this will in most cases be the important criterion for optimal delivery, and in cases where a constant release rate over a long period is required, the dissolution-limited approach in the next section can be applied.

To optimize this aqueous saturation approach, there should be a very low concentration of active-loaded microparticles in the outermost layer of the hydrogel region, otherwise a rapid transport of active to the skin directly from surface-layer microparticles could interfere with the desired release rate. There are at least three ways to ensure this. One way is to overlay, at a time point mid-way into the hydrogel crosslinking, a similar as-yet-uncross linked liquid solution that is microparticle-free, and crosslink this while the crosslinking of the original layer is still ongoing, such that the two layers crosslink together to form a single hydrogel, albeit heterogeneous with respect to microparticle concentration. While it is not always essential for the two layers to be covalently bonded together, it is preferred, in this invention. A second way is to apply an electric field, a temperature gradient, or a partial sedimentation in order to achieve a preferential distribution of microparticles away from the surface of the forming hydrogel. Just as electrophoresis is an established method to move particles against a concentration gradient, so is thermophoresis, though perhaps not as broadly known. A third method is to initially form the PNIPAM hydrogel in the form of small beads containing residual crosslinking groups, and then use the known thermophoresis of PNIPAM hydrogel beads to direct the functionalized hydrogel beads with a temperature gradient. The hydrogel beads will move away from warmer and toward cooler regions of water [see Wongsuwarn et al. (2012) Soft Matter 8:5857], where they can be crosslinked to each other and optionally with PNIPAM in the aqueous regions between beads.

Dissolution-Limited Release.

As discussed above, it is possible in this invention to achieve a constant or near-constant rate of release. This is of particular value for a drug that has a relatively low therapeutic index such that systemic levels should be kept as constant as possible over time, or when the diffusion-limited t^{1/2} profile would waste much of the active during the early-time high release rate.

Certain embodiments of the invention rely substantially, or even entirely, on the release characteristics of the matrix that is in direct contact with the active, rather than the PNIPAM hydrogel—to the extent that if desired, the thermoresponsive PolyNIPAM and the platelets can be completely omitted. The invention then takes the form of solid drug powder dispersed in a polymer matrix. It was realized in the course of this work that if such a polymer/active system were properly matched, and the egress of active from the matrix were substantially limited by the appropriate shape and coating of the polymeric particle, then near-constant rate of active release over an extended period of time could be achieved. This is now described in detail.

The mathematics of dissolution-limited release can be applied to optimize these embodiments of the present invention. Consider a rod-shaped polymeric particle containing dispersed active and coated with a “skin” over all but one end (face) of the rod. If A is the area of the face of the rod (cross-sectional area, for a perpendicular cut) and 1 its length, D is the diffusion rate of the active in the particle, K is the dissolution constant of the active in the polymer matrix, C₀ is the initial concentration of active/drug in the polymer (including dissolved and undissolved), and C_{S} is the saturation concentration of the active in the polymer, then the following equations hold:

$$Release rate = Q = \frac{d M}{d t} = {AC}_{s} \sqrt{DK}$$

$$Total mass of drug released = A {C}_{0} l$$

$$\begin{matrix}Duration of release = \frac{Total drug released}{Release rate} \\ = \frac{A {C}_{0} l}{( A {C}_{s} \sqrt{DK} )} \\ = \frac{( {C}_{0} / {C}_{s} ) l}{\sqrt{DK}}\end{matrix}$$

Since the release rate does not depend on the rod length 1 whereas the duration does, this means that the duration of release can be controlled by adjusting the length of the rod-shaped particles without affecting the rate of release. In short, the present invention provides not only for near-constant drug release, but also for independent control of release rate and duration of release. This is a particularly important advantage of the present invention because in practice, the choice of the polymer that forms the inner matrix will be driven by many factors other than D and K—cost, ductility, processability, crosslinking considerations, tack/adhesion, etc.—and one does not want to be restricted in polymer selection in order to meet kinetics requirements (D and K) without an easily adjustable parameter such as the aspect ratio of the particles.

Further control of rate and duration of release can be achieved by varying the non-occluded area over which release occurs. The skin can be applied to a reduced portion of the matrix polymer, leaving some fraction, say, 10% of the rod length non-occluded (in addition to the end face). Or, rather than cutting the rod perpendicularly, it can be sliced at an angle (analogous to a diagonal-cut string bean), increasing the exit area. With some polymers, in order to facilitate a sharp cut, the rods can be cut from a longer fiber while at reduced temperature, thus reducing the elastic behavior of fiber components. It should be noted that trying to reduce the effective non-occluded area by skinning this area with a permeable but reduced-diffusivity skin is not preferred, because this threatens the dissolution-limited characteristic that is crucial to constancy of release rate.

Near-constant release results from conformance to the following mathematical conditions:
the ratio D/(k·u) is greater than 1, more preferably greater than 10, and most preferably greater than 100;
the ratio 1K/D is less than 0.1, and more preferably less than 0.06;
the aspect ratio 1/d is preferably between 1 and 50, more preferably between 2 and 20, and most preferably between 2 and 10.

Noting that the ratio 1K/D in the second condition is nothing more than 1 divided by D/(K·u) when 1 is measured in centimeters, combining the first two conditions provides an easily visualized—as well as easily attained—relationship, namely: when D/(K·u)=1, then 1 must be less than 0.1 cm (1 mm), and when D/(K·u)=10, then 1 must be less than 1 cm, and if D/(K·u) is of order 100 then any practical value of the length is allowable. For the purposes of this disclosure, the term “rod-shaped” applies also to a fiber, recognizing that a long fiber held in a straight configuration is, in fact, rod-shaped, albeit with a high aspect ratio.

The aspect ratio 1/d, where d is the diameter of the rods, is preferably between 1 and 50, more preferably between 2 and 20, and most preferably between 2 and 10. For comparison, approximating the thin film in the disclosure of Chandrasekaran (U.S. Pat. No. 4,315,557) as a disc-shaped film (recognizing that the assumption of a round perimeter does not substantially affect the result here), the thickness 1 is stated to be “ . . . usually in the range of 50 to 1,500 microns (0.005 to 0.15 cm), and for even a small one-inch patch (i.e., d=1″, or 25,000 microns), that would put the aspect ratio in Chandrasekaran between about 0.002 and 0.06.

The ratio C₀/Cₛ should preferably be at least about 5, and more preferably greater than or equal to about 10.

The power of the approach can be illustrated thusly. If a crystalline drug is dispersed a polymer such that the following condition is obeyed:

Cₛ√DK˜10⁻⁹ gm/(cm²-sec),

and Ku<D, then this will result in near-constant release over an approximate 10-year period, given rod-shaped particles of diameter 1 mm and length 1 on the order of 1 cm. It should be noted that the 1=1 cm length, easily achievable in this invention, is an order of magnitude larger than the largest value of 0.15 cm recommended in U.S. Pat. No. 4,314,557. Indeed, in the present invention, for many applications is it preferred that the length 1 is greater than or equal to about 1 centimeter. It is clear that the length 1 of the end-releasing rods is the appropriate measure to compare with the thickness 1 of U.S. Pat. No. 4,314,557 since it represents the diffusional distance and since the dimensions at right angles to 1 determine the area over which release occurs.

As stated (though not adhered to) in U.S. Pat. No. 4,314,557 in order to achieve near-constant rate of release, one requirement is that the ratio D/(K·u), where u=1 cm, must be large—notwithstanding the fact that the polyisobutene/clonidine experiments reported in that disclosure did not obey this stated requirement. Preferably this ratio D/(K·u) is greater than 1, more preferably greater than 10, and most preferably greater than 100. For comparison with U.S. Pat. No. 4,314,557 this ratio for the polyisobutene/clonidine system in the experiment of that disclosure, the value of this ratio is approximately 0.1. In the present invention, it should be noted that if this ratio is greater than 17, then even with a rather large 1 of 1 cm, the ratio 1K/D will satisfy the relation 1K/D<0.06 that is required for near-constant release.

The “inner” polymer for the dissolution-limited embodiments, i.e., the matrix polymer in which the active is directly dispersed, is preferably selected from the following group: polysiloxanes (silicones), polyurethanes, polyanhydrides, polyisobutylene, elastin, natural rubber (polyisoprene), chloroprene, neoprene, butyl rubber, styrene-butadiene rubber (SBR), nitrile rubber, epichlorohydrin rubber, fluoroelastomers, polyether block amides, ethylene-vinylacetate (EVA), copolymers such as poly(styrene-b-isobutylene-b-styrene), ABS, etc. Partial phenyl substitution may be useful in the case of polysiloxanes to improve toughness. Especially preferred polymers from the standpoint of processing, in particular extrusion and related processes, are thermoplastic elastomers, such as styrenic block copolymers (TPE-s, such as Sofprene and Laprene), polyolefin blends (TPE-o), elastomeric alloys (TPE-v or TPV, such as Forprene), thermoplastic polyurethanes (TPU), thermoplastic copolyesters, and thermoplastic polyamides; these include in particular Arnitel (made by DSM), Solprene (Dynasol), Engage (Dow Chemical), Hytrel (Du Pont), Dryflex and Mediprene (ELASTO), Kraton (Kraton Polymers), and Pibiflex. Even water-soluble polymers can be used if they are extensively crosslinked, such as crosslinked polyhydroxyethyl methacrylate (PolyHEMA), gelatin, starch derivatives, polyethylene glycol, celluloses, natural gums such as gum arabic, gum tragacanth, xanthan gum, guar gum, gellan gum, dextran, etc.; in such an embodiment, the crosslinked polymer will typically be hydrated (to equilibrium swelling), so that the D and K parameters can be at least approximated by the corresponding values in water, which are often known accurately from prior knowledge (water being the most ubiquitous solvent on the planet, as well as in the lab). In cases where faster release rates are desired, a non-volatile and non-toxic solvent (or more generally, liquid) may be used to swell the matrix polymer. Tocopherol is an especially preferred liquid for this.

The “occluding polymer”, or “outer polymer”, or “skin” polymer that partially occludes the drug-in-matrix dispersion in the dissolution-limited embodiments, must be of low permeability to the active, be due to any combination of ultralow solubility and/or low permeability, the latter often being associated with a highly crystalline polymer, though high crystallinity is not necessarily required, if the polymer is in the glassy state near ambient temperatures. It is preferred that the melting temperature be low enough to allow easy processing. Preferred polymers for coating the solid dispersion are polypropylene, polyvinyl chloride, PTFE (non-porous), polyvinylidene fluoride (PVDF), PMMA, shellac, polycarbonate (viz., Lexan), polybutylene terephthalate, epoxy, polyethylene terephthalate (PET), high-density polyethylene, nylon, polyimide, celluloid, ABS, phenol-formaldehyde resin, and polystyrene.

While in principle it is possible for a lower surface energy outer polymer to creep over a higher energy inner polymer so as to occlude the desired non-occluded surface (e.g., the end of a rod), this can be easily prevented. One obvious way is to select two polymers with the correct order of surface energies, and this is rather simple because many if not most of the elastomers are of low surface energy (with polysiloxanes being prime examples). Another way is to take advantage of the high modulus of most of the polymers that one would naturally choose for the outer polymer, which are typically of high crystallinity, and arrange the processing conditions such that any tendencies to migrate are limited by the time spent in the molten state.

In selecting the matrix polymer in the dissolution-limited embodiments, one does not have to determine the individual parameters D, K and Cs. The same products that combine these individual parameters in the equations above also govern the release under more convenient experimental conditions, and can therefore be obtained by taking the slope of a measured release curve under those conditions. The resulting quantity can then applied to the conditions of the application.

Producing Dissolution-Limited Release Particles.

The multilayered polymer arrangements described herein can be produced by methods known in the art, most preferably co-extrusion. A powdered form of the active, obtained by wet or dry milling, controlled precipitation, spray-drying, etc., of the desired crystal size distribution, is first mixed into the matrix polymer, with elevated temperature if required to soften the polymer. Preferably the matrix polymer is either uncross linked at this point, or only lightly crosslinked; further crosslinking, if desired, can be applied at any stage subsequent to this mixing, and may even be engineered to occur during the mixing in a single operation (e.g., due to the elevation in temperature). While standard processes of intensive mixing, kneeding, or alternatively convective mixing or homogenizing (viz., at elevated temperatures), and the like can be applied, an interesting alternative is melt-blowing with an impacting stream of the powder, thus creating fiber contemporaneously with powder/polymer mixing. The matrix/active dispersion (which may at elevated temperatures in fact be a solid-in-liquid dispersion, or even an emulsion if the melting point of the active is low), is then extruded into the desired shape, typically a fiber, and the coating or “skin” applied either concomitantly using co-extrusion, or to the extruded fiber using standard methods of coating, such as spray coating, spray-drying, electrospray, fluidized bed coating, vapor deposition, etc. Roll-coating processes might be advantageous if the fibers are produced as a (woven or non-woven) web, which after coating would be subsequently broken or cut into segments of the desired length.

The polymer-rod dissolution-limited embodiments of this invention require a solid active ingredient, which may appear to be at odds with an emphasis on the utility of plants oils in this disclosure. However, one skilled in the art will recognize that in many if not most cases, the individual purified components of essential oils are often solids near ambient (room) temperature. For example, the liquid known as peppermint essential oil has as its predominant component menthol, which is a solid at room temperature. Menthol typical constitutes 50 to 80% of peppermint oil. Consider the case of 70% menthol: in such a peppermint oil, the 70% menthol component is accompanied by 30% of “other ingredients”, generally quite similar in molecular structure to menthol, but different enough that these minor ingredients act to lower the melting point of the menthol. Quite broadly, this melting point depression effect is very common in plant oils, and means that many of the benefits from essential oils discussed in this disclosure can in fact be effected by solid actives, which are suited for the polymer-rod dissolution-limited approach disclosed herein.

Application to Substrate.

It will generally be highly desired to establish a strong connection of the controlled-release particles to a substrate. The substrate upon which the particle (polymer/active dispersion, or hydrogel bead) is deposited may be metallic, ceramic, polymeric (glassy, semicrystalline, or elastomeric), or composite, though in most of the embodiments discussed herein this is a fabric. An example of a metallic substrate would be finger-worn jewelry such as a ring, for medicating against arthritis. Preferably the substrate conforms to one part of the body, and therefore orthopedic cast and splint materials are particularly useful, as well as wound dressings, and ordinary tight-fitting fabrics such as socks, hats, face/ski masks, scarves, tiaras, chokers, skullcaps, undergarments, skin guards, wrist bands, arm bands, knee pads, bras, nylon stockings, athletic supporters, robes, neck bands, head bands, ear muffs, gloves, diapers, poultices, facial masques, paraffin glove, joint braces, pillowcases, blankets, sheets, and furniture coverings. Preferred substrates in this invention are fabrics, both woven and nonwoven, and foams such as polyurethane foams. Most preferred substrates are fabrics and foams in the form of socks, pillowcases, gloves, and wound dressings, with the most preferred material being bamboo fabric.

For hydrogel embodiments meant to release from a substrate, perhaps the simplest methods for attaching hydrogel films or beads to a substrate are through the use of adhesives and binders. Generally, any adhesive or binder that completely covers the hydrogel surface area will severely restrict active diffusion out of the hydrogel. Therefore it is advised, and preferred, in the use of adhesives and binders to substantially restrict the adhesive or binder so as to avoid covering at least one area of each hydrogel bead or film where release can readily occur. In most cases, this will most preferably be the area that is in contact with the skin or other tissue; however, in cases where the release of active vapors (viz., for inhalation) is favored over release of liquid, then the uncovered area could more preferably be chosen to be area that is not in contact with skin. In the first case, a preferred method of applying the hydrogel, whether beads or films, is to apply a thin layer of adhesive or binder to the substrate and, before curing, deposit the hydrogel onto the adhesive. In most cases it is desired that the thickness of adhesive be significantly thinner than the hydrogel, since otherwise it could restrict the swelling and de-swelling of the hydrogel. Furthermore, it is preferred that the adhesive film be “patchy”, that is, it should not occlude the substrate and destroy breathability, and one simple way to achieve this is to spray the adhesive on the substrate with significantly less than 100% coverage. If the substrate is highly porous and the adhesive does not have a positive spreading coefficient on the substrate, then “painting” the adhesive on the substrate may leave sufficient porosity to allow for sufficient breathing. In the case of a hydrogel film of large area, it may be possible to apply the adhesive to one side of the hydrogel film, and pasting this on the substrate, again with due respect for the breathability of the final product. Preferred adhesives, based largely on compatibility with hydrogels and on crosslinking behavior (to minimize dissolution), are casein glue, collagen glue (also known as bone glue), balsam, methyl or nitro cellulose, starch (e.g., library glue, wheatpaste), acrylic, resorcinol glue, polyester resin, polyurethane (such as Gorilla glue), hide glue, epoxy glue, PVA glues including Elmer's and carpenter's glues, polyvinyl chloride emulsion (PVCE), PVP-based glues, and xanthan and tragacanth gums.

Another method that is relatively simple to implement, though perhaps not to optimize, is to use another fabric or polymer film as a liner to retain the hydrogel. In other words, the hydrogel lies between the main substrate and the liner. The liner can either be on the side that is in contact with skin or tissue, in which case it must have ample permeability to the active so as to allow the desired delivery, or the fabric can be on the skin contact side with the liner on the “outside”. In the latter case, the porosity in the substrate could provide the necessary permeability to active, and may even allow for direct skin-hydrogel contact if the substrate is sufficiently thin. It is important to recognize that if a “breathable” treated fabric is desired, as will most often be the case, then the liner must be breathable itself; however, this can if desired be performed so as to avoid introducing pores that compromise the retention properties of the liner, by using polymers with extremely fine porosity or with inherent permeability—for example, a thin silicone film can be permeable to oxygen, as well known in the art. Another method that retains breathability is to apply the liner in patches, with spaces between.

Covalent bonding of the PNIPAM-based polymer to the substrate has many advantages, and this can be accomplished by a low concentration of reactive groups on the PNIPAM-based polymer. These reactive groups should be selected so as to react with, and form covalent bonds with, the substrate material, and most typically are incorporated into polymer by using an acrylic monomer that is decorated by other reactive groups besides the acrylate group. For example, if the substrate has amino groups, then an acrylic monomer such as acrolein containing one or more aldehyde groups can provide for a substrate-binding reaction that is compatible with water.

Dissolution-limited release polymeric particles of the invention may, as discussed herein, be partially coated or “skinned” with an active-impermeable polymer, called Polymer A or the “occluding polymer” herein, and one of the distinct advantages of this approach is that by choosing Polymer A to have reactive groups for covalent attachment to a substrate, the propensity for the substrate attachment to interfere with active release is minimized. After all, the non-occluded region where release occurs would be neither involved in the binding reaction nor (in general) occluded by the fabric, in this approach.

If the substrate material does not nominally contain suitably reactive groups, then several approaches can be used to create reactive groups in the material, particularly at the surface. Glow discharge, corona discharge, gas atmosphere plasma, flame plasma, atmospheric plasma, low pressure plasma, vacuum plasma, glow-discharge plasma, and plasma etching are all established methods for introducing reactive groups at surfaces. Other methods include exposure of the substrate material to strongly acidic or basic solutions, or to solutions of reactants such as peroxides, or compounds that react with carbonyl groups that are ubiquitous in polymers such as diazomethane, Grignard and Wittig reagents, primary and secondary amines, dilithio oximes, sodium alkynides, and hydrides, etc. In the case where the substrate is a polysaccharide such as a cellulose, reactants that react with such polymers are well known to one skilled in the art, so as boron-based reactants, etc. Alternatively, the substrate material can be formulated so as to contain the desired reactive groups.

In addition to the attachment methods described above, one can physically embed the controlled-release particles of the invention either in the interior of the individual fibers, or in the interstices between fibers. If the fibers are electrospun, then methods known to one in the art can be used to embed particles in the fiber core. Alternatively, a method for depositing fibers that is relevant to this approach is given by U.S. Patent App. No 2008/0242171 to Huang et al., which states: “For example, a nonwoven web or other porous scrim material, such as a spunbond web, a melt blown web, a carded web or the like, can be placed on the collector and the fiber deposited onto the nonwoven web or scrim. In this way composite fabrics can be produced . . . ”. Thus in applying that methodology to the present invention, the hydrogel is applied contemporaneously as the support mesh is produced.

Application of the Invention.

The present invention can be applied to a range of conditions. The following are representative and illustrative, though certainly not all-inclusive. For each of the application areas shown below, particular actives for that application are given in the Table below.

For the PNIPAM embodiments of this invention, in addition to diffusion-driven transport, other methods can be used to promote transfer of active from the hydrogel to the target tissue. These include electrophoresis (as in iontophoresis), directed fluid flow across the gel, hydrophoresis, magnetophoresis, ultrasound/sonication, and the use of one or more solvent-rich layers situated between the gel and the target tissue (e.g., skin, mucosal tissue). In the latter, several arrangements are possible: A) hydrogel beads can be suspended in a water-immiscible solvent; B) hydrogel beads or a hydrogel film can be situated so as to straddle the interface between a water-rich solvent system and a water-immiscible solvent; and C) a water-immiscible solvent can be situated between a hydrogel film and the target tissue. The water-immiscible solvent layers in these embodiments can serve several purposes, namely accelerating or controlling the diffusion rate of active out of the gel (i.e., the release), lubricating against friction from the gel, enhancing active/drug absorption into the target tissue, enhancing systemic active/drug delivery, and preventing desiccation of the gel. It should be clear to one skilled in the art that the viscoelastic properties of the water-based and/or water-immiscible solvent systems can be adjusted by the use of additives such as polymers, clays, etc.

For the dissolution-limited polymer rod embodiments of the invention, certain more industrial applications can be envisioned that do not involve fabrics. One such application is for the slow-release of antimicrobial actives—in particular antifungals—into paint, or other coatings such as lacquers, varnishes, primers, stains, shellacs, sealants, and enamels. As illustrated in one of the above equations, it is feasible to screen for the proper selection of matrix polymer and adjust the rod dimensions such that the dissolution-limited release occurs steadily over a ten-year period, in this case releasing into dried paint, or other coating. As is characteristic of dissolution-limited release, the rate of release is determined by events (viz., active dissolution) that are completely internal to the polymer matrix, and thus nearly completely unaffected by the environment outside the particle, and this means that the active in this example will also release at this same 10%-per-year rate in the paint can prior to application of the paint. Thus, if the paint sits on the shelf of a hardware or paint store for, say, 1 year, then 10% of the active will have been released, making for 9 years of release in the applied/dried paint, which may be entirely acceptable. In such a product, it may not be necessary to break long fibers (resulting from the extrusion, melt-blown, or other processing) into small rods, as the agitation that is routinely applied to cans of paint (and other coatings) just prior to application might be sufficient to break down the fibers into rods which have “naked”, non-occluded ends. It should be noted that typically in such applications, the antimicrobial, antioxidant, plasticizing, intumescent, optically-active, catalytic, cleansing, etc., active is most preferably released into the coating, so that release predominantly to the outside of the dried coating is not required, and there is no need for particle releasing surfaces to be at the outer surface.

For most of the applications discussed below, either the PNIPAM hydrogel approach or the dissolution-limited polymer rods can be used. For substrates, e.g., textiles, that may spend a substantial fraction of their functional lives in hot water environments such as laundry, the PNIPAM embodiments may be more favorable. However, at the time of the writing of this disclosure, cold-water laundry detergents are becoming increasingly prevalent and thus for many if not most applications, the dissolution-limited polymer rods will be preferred.

Some surprising advantages come from the use of the present invention. Large dosages, of several grams or more per dosing, that are problematic through pills and other dosing forms, can be administered through skin-contacting clothing in a way that is convenient, private, and even fashionable. Forgetful patients, such as schizophrenics, children, elderly, Alzheimer's or pre-Alzheimer's sufferers, and the like can be assured of taking their medication (i.e., increased “compliance”) by virtue of simply lying on a pillow at night, or putting on their socks or another article of clothing. Long-term use of a transdermal approach can be used without engendering the downsides of occlusive, and/or adherent, patches or bandages, downsides that can include angiogenesis. Site-specific medications can be delivered to sites, such as the feet or hands, which are well-suited for delivery via clothing (socks, gloves) but not for other local vehicles. From the other side of the coin, current fabric-based products of purported medicinal value, such as diabetic socks for example, which have not been provided with the obvious medicaments due to washing requirements, can now be medicated and yet still remain fully washable. Drugs and actives that are not themselves patentable (e.g., because of prior use or premature publication) and would therefore not be pursued by pharmaceutical companies can be formulated as patent-protected fabric-based drug products. And in a world that is ever increasing the demand for products of high design and fashion, the present invention can provide delivery of medicament both locally and systemically through fashionable fabrics.

Skin Conditions.

For application of active(s) to portions of skin suffering from abnormalities or for cosmetic improvement, the present invention offers direct skin contact, localizable coverage, washing machine compatibility (“washability”), constant rate of release or day-to-day uniformity, continuous coverage through the night if desired or, as a patch, throughout the 24-hour clock. Actives for particular skin conditions are shown in the Table below, such as tea tree oil for acne, eczema, psoriasis, etc. In addition to acne, other skin conditions for which the invention is particularly useful include rashes, skin allergies, folliculitis, impetigo, erysipelas, cellulitis and dermatitis.

In applications that can be considered therapeutic, cosmeceutic, cosmetic, or other descriptor, embodiments of the PNIPAM hydrogel and polymer-rod approaches disclosed herein can improve skin condition and appearance by the release—most preferably at a relatively constant (uniform) release rate—of vasodilators, rubefacients, ceramide, emollients, dermoprotective, lipolytic, or epithelializing compounds.

The invention can be of particular utility in medication- or antimicrobial-releasing socks, because socks must be washed so frequently, and the need is inherently high due to the relatively high rate of foot- and sock-related disorders, risks, and inconveniences, such as offending odors and the associated risks of infections (not only bacterial but also fungal and viral), and more serious risks faced by the growing incidence of diabetes.

In addition to acne, eczema and psoriasis, the following conditions—which are far less common and therefore less likely to be the focus of extensive work at traditional pharmaceutical companies—may be treatable, or preventable, with embodiments of the present invention: scleroderma (which often leads to Raynaud's syndrome), neutrophilic dermatosis, urticaria, xeroderma-pigmentosum, Goltz syndrome, recessive dystrophic epidermolysis bullosa, Harlequin ichthyosis, hypertrichosis, Morgellons disease, dermatofibrosarcoma protuberans, and infections such as Human papilloma virus (HPV). Scleroderma comes in non-systemic and systemic forms, and while the invention is particularly well suited for treating the non-systemic form, for example with a fabric that would release an active oil extract from Salvia miltiorrhiza (Danshen) and/or from Capparis spinosa, it may be effective against the systemic form as well. Salvia miltiorrhiza and Capparis spinosa work against scleroderma in two distinct mechanisms, so that a combination of the two oils may be particularly efficacious.

Wound Dressings.

In a related application area, the present invention can provide for wound dressings that can be either spray-applied—i.e., by applying a liquid formulation that is crosslinked after spraying—or applied with a hydrogel that is fully formed. The former spray-application method clearly is well suited for highly irregular wounds, and also has the advantage of being an extremely mild method for the tissue. Whether the initial drug-releasing is from spray-applied particles or beads of the invention, or a fabric-based embodiment of the invention, the invention can provide wound dressings that are non-adherent, non-occlusive for oxygen transport, and non-irritating. Bamboo fabric is a preferred fabric as is it not only substantially non-irritating and non-allergenic (with allergic responses occurring only very rarely), but is a “breathable” fabric, and importantly, direct PNIPAM hydrogel-skin contact can be almost entirely non-adherent, meaning that removal and re-application of such a dressing could be pain-free and, more importantly, not disruptive to healing tissue. Wounds for which the present invention is particularly well suited include chronic wounds, such as malignances, persistent infections (e.g., gangrene), decubitis and diabetic ulcers, and other ulcers of traumatic, venous, or ischemic origin. While the invention can certainly be used as a primary dressing, it can also be effective as a secondary dressing, delivering medicament through the primary dressing.

One potential application related to wound dressing is that of an insert or lining to a cast, splint, sling or brace. There are over 6.8 million broken bones just in the US every year. In the case of individuals treated for scoliosis, patients must wear a full body cast and lie in bed for 3-6 months. There are many common negative issues associated with wearing casts for prolonged periods of time, including but not limited to, allergic reactions, skin sores, infections, joint stiffness, muscle loss, offensive odor, burns and compartment syndrome which greatly limits blood flow. All of these negative side effects could potentially be effectively treated or mitigated by our invention. This application of the invention would be in the form of an insert or lining to a cast, splint, sling or brace. The cast/insert system could be designed such that the insert could be removed, daily if necessary, for washing without interfering with the supportive and protective functions of the cast or brace. The insert could provide release of antimicrobials, growth factors, analgesics, and skin toning/cosmeceutical actives, and release medicaments or essential oils designed to increase blood circulation.

Crosslinking can be accomplished by any of the means listed above, but in addition, in this application area it may prove advantageous for the crosslinking to be catalyzed, or indeed complemented, by naturally polymerizing compounds in the body, particularly those associated with natural wound processes such as clotting. Among other things, this could be done so as to end up with interpenetrating networks (IPNs) of PNIPAM and biopolymer such as fibrin, thus improving tissue compatibility.

One skilled in the art will be familiar with the types and identities of actives that are beneficial for treatment of wounds: growth factors, clotting factors, local anesthetics, steroids, vitamins, minerals, antimicrobials, or in milder wounds antiseptics and bacteriostats.

Sleep/Relaxation/Pain.

The present invention can deliver sleep/relaxation-aiding actives both into the bloodstream through release into the skin, and into the brain through the trigeminal neural pathway via nasal inhalation. Due to the fact that compounds and oils from nature that induce relaxation often have analgesic action as well, viz., due to action at one or more opioid receptor, the invention can be applied to release these actives and—potentially with combined transdermal and trigemical (inhalation) delivery routes—achieve a synergistic combination of anxiolytic and analgesic actions. One particularly useful combination of two actives is the combination of lavender and Melissa essential oils. Plant essential oils that are purported analgesics include lavender, wintergreen, Roman chamomile, marjoram, peppermint, rosemary, thyme, vetiver, helichrysum, ginger, lemongrass, copaiba (copal), and balsam fir. Specific fractions or components of these oils, such as menthol, can be used as well, particularly if they have substantial volatility. Preferably the vapor pressure of the active at 35° C., for inhalation/trigeminal neural pathway delivery is equal to or greater than 0.01 Torr, more preferably greater than about 0.1 Torr, and most preferably greater than about 0.5 Torr. A drug with lower vapor pressure than this may still be practical if the potency of the drug is very high, such as with carfentanil.

Extracts of the following plants have been reported in the literature to have central-acting analgesic activity, and these could be incorporated into the present invention for relief of pain and, in many cases, for relaxation as well: Abutilon indicum, Acacia ferruginea, Acacia nilotica, Achillea ageratum, Acicarpha tribuloides, Aconitum carmichaelii, Aconitum flavum, Aconitum japonicum, Acorns calamus, Adansonia digitata, Afrormosia laxiflora, Agastache sinense, Ageratum conyzoides, Albizia lebbek, Alhagi maurorum, Aloe vera, Amelanchier ovalis, Anacardium occidentale, Anchomanes difforms, Annona squamosal, Apium graveolens, Araujia sericifera, Astragalus siculus, Baphia nitida, Berlinia grandiflora, Brassica rapa, Buddleja cordata, Bupleurum chinense, Cadia rubra, Caesalpinia ferrea, Calotropis procera, Cannabis sativa, Canthium parviflorum, Caralluma tuberculata, Carthamus tinctorius, Cedrus deodara, Celastrus paniculatus, Centella asiatica, Chasmanthera dependens, Chelidonium majus, Chrozophora verbascifolia, Cinnamomum zeylanicum, Citrullus colocynthis, Clematis chinensis, Cleome viscose, Clerodendrum infortunatum, Clitoria ternatea, Cocculus pendulus, Commiphora molmol, Cordia francisci, Cordia martinicensis, Cordia myxa, Cordia ulmifolia, Cucumis trigonus, Culcitium canascens, Curcuma zedoaria, Cuscuta chinensis, Cyathea nilgirensis, Cymbopogon schoenanthus, Cystoseira usneoides, Datisca cannabina, Desmodium canadense, Dioclea grandiflora, Diodia scandens, Dolichos falcatus, Ducrosia ismaelis, Egletes viscosa, Elaeagnus kologa, Elaeocarpus canitrus, Eriobotrya bengalensis, Ervatamia coronaria, Eryngium foetidum, Eucaluptus camaldulensis, Euphorbia hirta, Fagraea racemosa, Ficus glomerata, Foeniculum vulgare, Ganoderma lucidum, Genista patens, Glaucium flavum, Harpagophytum procumbens, Hedera rhombea, Heracleum hemsleyanum, Hibiscus sabdariffa, Himanthalia helongata, Himulus lupulus, Hypericum calycinum, Hypericum perforatum, Inula crithmoides, Inula viscosa, Ipomoea leari, Irvingia gabonensis, Juniperus oxycedrus, Laminaria achroleuca, Lantana camara, Lawsonia inermis, Ledebouriella seseloides, Lepidium sativum, Leucas aspera, Leucojum aestivum, Ligusticum sinense, Lippia alba, Lippia geminate, Luvunga scandens, Lycopodium clavatum, Lysimachia christinae, Maesa ramentacea, Melaleuca elliptica, Melaleuca styphelioides, Mentha piperita, Mikania cordata, Morinda citrifolia, Moms alba, Mucuna pruriens, Myrica nagi, Myrtus communis, Nepeta caesarea, Nepeta italica, Neurolaena lobata, Nigella sativa, Nyctanthes arbor-tristis, Ocimum sanctum, Oplopanax elatus, Origanum onites, Paeonia moutan, Panax ginseng, Pancratium maritimum, Paullinia cupana, Peganum harmala, Persea Americana, Photinia serrulata, Phyla nodiflora, Phyllanthus niruri, Phyllanthus sellowianus, Phyllanthus tenellus, Phyllanthus urinaria, Pimpinella anisum, Pinus koraiensis, Piper abutiloides, Piper cincinnatoris, Piper lindbergii, Piper longum, Piper methysticum, Piper umbellatum, Piscidia erythrina, Platycodon grandiflorum, Polygala cyparissias, Polypodium vulgare, Pongamia pinnata, Portulaca grandiflora, Portulaca oleracea, Prunus spinosa, Psammosilene tunicoides, Psidium pohlianum, Psychotria brachypodia, Psychotria colorata, Pterocarpus indicus, Ptychopetalum olacoides, Pycnocomon rutaefolia, Quercus infectoria, Quercus lineata, Randia siamensis, Ranunculus japonicas, Rhamnus procumbens, Rhazya stricta, Ricinus communis, Roylea elegans, Salvia haematodes, Santolina chamaecyparissus, Saussurea involucrate, Scabiosa atropurpurea, Senna italic, Serjania communis, Sida cordifolia, Sideritis mugronensis, Siphocampylus verticillatus, Stephania dinklagei, Stefania wightli, Strychnos nux-vomica, Synedrella nodiflora, Tabebuia chrysotricha, Tabernaemontana pandacaqui, Tamarix milotica, Taraxacum officinale, Teclea nobilis, Tecomella undulate, Teucrium carthaginense, Theobroma leiocarpa, Thymus vulgaris, Tillandsia usneoides, Tinospora cordifolia, Tinospora crispa, Torresea cearensis, Trachelospermum jasminoides, Trema guineensis, Trianthema portulacastrum, Tribulus terrestris, Trichilia catigua, Trigonella anguina, Trigonella foenum-graecum, Typhonium giganteum, Urtica dioica, Valeriana jatamansi, Vernonia condensate, Viola mandshurica, Vitex negundo, Zingiber officinale, and Ziziphus jujube.

Medicated Contact Lenses.

The aging of the population is leading to an increase in such eye disorders as macular degeneration, cataracts, various inflammatory conditions, etc. A promising method of treatment involves the use of soft contact lenses that deliver medicines, and in such an application, the present invention can be used to provide for washability of the contact lenses in warm or hot aqueous cleansing solution, with acceptable or minimal loss of active. As in many of the applications discussed herein, it is understood that instructions for use of these products should include statements that the washing should always be at warm or, in some cases, hot temperatures, since below the collapse temperature a washing operation could result in unacceptable (and easily preventable) loss of active. The critical collapse temperature Tc in a contact lens application of the invention would need to be higher than about 39° C., and instructions for cleaning would need to require cleaning water temperatures greater than this Tc. The invention can be used, among other things, to aid in the use of contact lenses, or to treat conditions of the eye attendant to their use, and to treat dry eye, aqueous deficiency, mucin deficiency, meibomian gland dysfunction, neurogenic dry eye, and inflammation associated with dry eye and to protect and coat the ocular surface. The invention could also be used in the contact lens format to accomplish systemic delivery of a drug through the eye, this route being known as intravitreal. Washing of contact lenses on a daily basis is well-established to be critical to eye health as well as contact lens functionality. Actives of particular value include steroids and steroidal anti-inflammatory agents, including fluorometholone, prednisolone acetate, prednisolone phosphate and especially dexamethasone; antiinfectives such as bacitracin, erythromycin, polymyxin, neomycin, and tobramycin, ciprofloxacin, gentamycin, sulfacetamide, and combinations thereof; cyclosporin, mycophenolate mofetil, triamcinolone; and local anesthetics for pain relief such as bupivacaine, lidocaine, procaine, tetracaine, mepivacaine, etidocaine, oxybuprocaine, cocaine, benzocaine, pramixinine, prilocalne, proparacaine, ropivicaines, chloroprocaine, dibucaine, as well as nutriceuticals such as Vitamin E, Vitamin A, Vitamin D, zeaxanthine, and carotene. Specific classes of compounds that can be incorporated and delivered include demulcents, emollients, lubricants, vasoconstrictors, antibiotics and antiseptics, antihistamines, immunosuppressants, local anesthetics, antiallergics, antifungals, vasoprotectants, anticoagulants, mucolytic and proteolytic compounds, antiglaucoma drugs, and anti-inflammatories, anesthetics, anti-helminthic, analgesics, steroids, non-steroidal inhibitors of the inflammatory cascade, anti-neoplastic, anti-angiogenic, calcineurin inhibitors, anti-ocular hypertensives, antivirals, antibacterials, neuroprotectants, anti-apoptotics, medications for dry eye, pupil dilating medications (mydriatics and cycloplegics), ocular decongestants, anti-oxidents, photosensitizers, photodynamic therapy agents, mast cell stabilizers, monoclonal antibodies, quinolone antibiotics, and intra-ocular pressure lowering agents. Specific ophthalmic pharmaceutical actives in addition to the above which may be incorporated in the present invention are: acetazolamide, amikaci, anecortave, antazoline, apraclonidine, atropine sulfate, azelastine, azithromycin, bacitracin, bacitracin zinc, betaxolol hydrochloride, bimatoprost, brimonidine, brinzolamide, bupivicaine, carpbachol, carteolol hydrochloride, ceftazidime, ciprofloxacin hydrochloride, clindamycin, cromlyn, cyclopentolate hydrochloride, denufosol, dexamethasone, dexamethasone sodium phosphate, diclofenec sodium, dipivefrin hydrochloride, diquafosol, dorzolamide, doxycycine, edetate sodium, emadastine, epinastine hydrochloride, epinephrine, erythromycin, fluocinolone, 5 fuoruracil, fluoromethalone, fluoromethalone acetate, flurbiprofen sodium, fomivirsen, ganciclovir, gatifloxacin, gentimicin, gramicidin, imopenemn, ketotifin, ketrolac tromethamine, latanoprost, lerdelimumab, levocabastine, levofloxacin, levubunolol hydrochloride, lidocaine, lodoxamide, lotoprednol etabonate, medrysone, methazolamide, metipranolol, mitomycin, moxifloxacin, naphazoline, nedocromil, neomycin, ofloxacin, olopatadine, oxacillin, oxymetazoline hydrochloride, pegaptanib, pemirolast, pheniramine, phenylephrine hydrochloride, photofrin PIR 335, pilocarpine hydrochloride, polymixin B, prednisolone acetate, prednisolone sodium phosphate, proparacaine, ranibizumab, rimexolone, scopolamine hydrobromide, sulfacetamide sodium, tetracaine, tetrahydrozoline hydrochloride, timolol, timolol maeate, tobramycin sulfate, travoprost, triamcinolone acetonide, trimethoprim, tropicamide, unoprostone, urea, vancomycin, and verteporfin. Also suitable are derivatives, analogs, and prodrugs, mixtures and combinations thereof.

Fungal Infections.

Fungal infections of the skin are notoriously long-lasting, and compliance with an antifungal spray can be poor due to the need for daily application in the harried early morning time. An antifungal-medicated piece of clothing that is washable could provide for long-term application to the site of infection without requiring any compliance on the part of the user, beyond the normal washing of the fabric that is required in any case. With, say, 4 or 5 pairs of medicated socks, one could maintain continuous application of the active to the site during all waking hours of the day, and even at night if desired, without any conscious effort other than donning the designated socks each morning.

Disorders of the Respiratory System.

Vapor-releasing salves are notoriously short-acting, and are not well suited for constancy of release. On the other hand, prior art patches capable of constant release rates are unsightly and even disfiguring. The present invention can overcome these by providing a sufficiently sophisticated delivery system for constancy of release which is nevertheless in the format of a fully functional (viz., washable) article of clothing, such as a scarf, cap, veil, woven necklace, choker, neck band, ear muffs, or other headwear. Trigeminal neuropathy, also known as “the suicide disease” due to the excruciating pain it causes, may be treatable by using an embodiment of this invention that releases pain-numbing vapors such as menthol at a more constant rate than salves. Other conditions possibly treatable with such an approach include nasal congestion, emphysema, sarcoidosis, pleural effusion, pulmonary edema, pulmonary hypertension, pneumonia, tuberculosis, various infectious diseases, respiratory irritation (e.g., from breathing polluted air), and non-productive coughing.

Nutrition/Nutriceuticals.

It is difficult for a pharmaceutical company to justify expending resources developing and marketing nutritional compounds, both because most are unpatentable and because that is not the focus of a pharmaceutical company. For example, the inventors are not aware of any marketed transdermal patch that delivers a nutriceutical. However, it does make business sense for a company focused on nutritional and nutriceutical compounds to embed these in everyday-use fabrics, and the large surface areas for transdermal delivery made possible by the invention could allow for delivery of larger doses than would be possible for traditional transdermal patches.

Pharmaceuticals, Transdermal Delivery.

Considerable instruction has been provided herein for producing washable, medicated materials for delivery of drug—including at a near-constant rate—to the skin, which with many drugs translates into systemic delivery, i.e., transdermal delivery to the bloodstream. Nicotine, fentanyl, methylphenidate, scopolamine, nitroglycerine, rivastigmine, clonidine, Vitamin B12, estrogen and testosterone are some examples of drugs that are currently delivered transdermally through medicated patches, which are, of course, not washable, and thus must be discarded when dirty. Drugs requiring daily (or near-daily) application could benefit from the present invention; for example, with children's ADHD, exposure to dirt of all forms is of course to be expected for a (hyperactive) child, and a washable, reusable patch could be an advantage. Furthermore, if the present invention is used in the form of an article of clothing, particular one that is fairly tight-fitting such as a sock or cap, then it becomes possible to eliminate the need for adhesives, which are essentially required for traditional transdermal patches and present a range of practical issues. The invention could also be used to deliver drugs systemically through mucosal membranes, a route known as transmucosal.

Scented Fabrics.

It is within the scope and spirit of this invention for the “active” to be one that improves the quality of life through the steady release, even through many washes, of a pleasant and social aroma, including even the possibility of pheromones. The designs discussed elsewhere herein for promoting release into the air (discussed above in relation to inhalation-based delivery) would be preferred for such an application. Many of the essential oils listed and discussed herein are well established as pleasing aromas or even as perfume components. The more sophisticated embodiments discussed herein that yield more nearly constant release rate could be used to create textiles, such as dresses and scarfs, which do not suffer from the relatively short action of a single application (spray) of perfume, and in fact do not require any action on the part of the customer.

Detoxification.

As mentioned herein, a crosslinked-PNIPAM and platelet-containing hydrogel, loaded with liquid-containing or liquid crystal-containing droplets or particles, can be used to remove toxins from a tissue such as, though not limited to, skin. In the case of local toxin removal, such as with a bug or snake bite, then a fairly small surface area of material of the present invention might be fine for removing toxins from a particular site. In contrast, it should be clear to one with skill in the art that facile removal of a systemic toxin generally requires a substantial surface area, and in such a case, a toxin-removing article of clothing incorporating the present invention can be far less intrusive and unsightly than a large-area patch. In such an embodiment of the invention, the main purpose of the collapse-mediated diffusional block triggered by the hot water of a wash cycle might actually be to minimize the loss of microparticles or microdroplets during washing.

Drug/Nutriceutical Delivery to Infants and Toddlers.

Delivery of drugs and even some nutritional supplements to infants and toddlers is generally a challenge due to swallowing/coordination limitations and taste intolerance. The present invention provides convenient means for overcoming these delivery challenges, by incorporating medicament- or supplement-releasing embodiments of the invention into and onto commonly-used (and frequently-washed) items such as pacifiers, milk/formula bottles, stuffed animals etc. Hydrophobic actives, in particular, will in general be released more rapidly into milk or formula than into water, and milk, particularly flavored milk, can mask the taste of medicaments, providing for relatively high dilutions without increasing total fluid intake.

Gloves releasing circulation-improving compounds or oils (vasodilatory, rubifacient) and/or local anesthetic compounds for treatment or prevention of Raynaud's disease and related conditions.

Athletic garments and undergarments releasing one or more of the following: performance-enhancing actives; aspirin and/or capsaicin for relief of pain or cold; natural oils, balms, creatine, glutamine, citrulline malate, beta-alanine, branched-chain amino acids, for muscle recovery or muscle stimulation;
Handkerchiefs releasing cologne or perfume, antimicrobials, and/or vitamins.
Paint . . . anything related to rain. Outdoor stuff . . . rain washing away.
Skin whitening
Oil/shale mining
Fertilizer
Paint

Specific Applications.

Examples of applications of the present include the following, particularly where washable, reusable properties are a significant benefit:
Acne-fighting pillowcases; Cosmeceutical (e.g., anti-wrinkle) pillowcases; Anti-bacterial pillowcases/gowns for use in hospitals, etc.; Insect repellent sheets/clothes; Fire retardant clothing/sheets; Appetite suppressant-delivering garments or patches; Hair nets or head gear with actives for hair care, dandruff prevention, treating dry itchy scalp, treating lice and other hair conditions, etc.; Hair growth-promoting pillowcases, hats, caps, beanies, etc.; Aphrodisiac pillowcase/sheets; Textiles, patches or dressings releasing actives for sun burn relief; Scaffolds for tissue growth/engineering; washable ECG and EEG electrodes; Medicated gels for iontophoresis; Absorbent pillowcase/sheets that can reduce levels of prevalent early morning chemicals; Cortisol-absorbing fabrics to combat collagen breakdown; Cologne- or scent-infused clothes/sheets/pillowcases; Dermatosis- and scleroderma-fighting pillowcases or sheets; Cooling or heating sheets/pillowcases/clothes (e.g., using cooling oils such as eucalyptus, or heating oils such as capsicum oleoresin; Sleep-aiding pillowcases and sheets; Detoxification with liquid or liquid crystal microparticle-containing sheets, pillowcases, headwear, etc.; toxin-absorbing microparticles can help with hangovers, remove excess alcohol and aldehydes from the body; similar approaches could also work for toxic drugs or overdoses; Reduce cigarette cravings, reducible levels of nicotine in clothing, pillowcase or sheets; Vitamin D-delivering pillowcase, particularly in wintertime in northern climates where seasonal affective disorder is a problem; Hormone-delivering garments or patches, for delivering either locally beneficial steroids such as dexamethasone, or systemic hormones such as testosterone, estrogen, etc.; Moisturizing sheets and clothes, pillowcases, particularly for moisturizing skin over long periods of time; Diabetic socks, medicated for delivering OA or RA-fighting oils, and for improving circulation; Men's underwear incorporating drugs for intercavernous delivery (penile delivery); Garments and dressings for blister/sore prevention and treatment; Medicated socks for prevention and treatment of athlete's foot and other infectious diseases of the feet; Medicated or scented socks to prevent malodors; Medicated wound dressings that are breathable yet maintain active throughout showering and bathing; Toxin- or exudate-removing wound dressings that are breathable yet maintain active throughout showering and bathing; Poultices that release medicament after cooling; Antimicrobial-medicated fabrics and wound dressings for the treatment of fungal, bacterial and viral infections; Fabrics, in particular bedding, for inhalation-based delivery of actives (potentially involving the trigeminal neural pathway) for depression/motivation issues, sleep disorders and simple insomnia, nausea, headaches, muscle aches, migraines, pain relief, and psychiatric conditions; Fabrics, in particular bedding, for lung delivery of actives for treatment of asthma, coughs, colds, allergies, COPD, and lung infections, sleep apnea, and reducing snoring; Medicated fabrics, patches, and dressings for treatment and prevention of cold sores, dermatitis, eczema, psoriasis, warts, boils, cystitis, hives, bruises, and insect bites and stings (mosquitos, spiders, ticks, bees, hornets, bumblebees, yellow jackets, wasps, fire and harvester ants, conenose bugs, horseflies, deerflies, Tse-Tse flies, spiders, ticks, bedbugs, black flies, etc.); Fabrics or patches carrying scents that repel insects; Fabrics and wound dressings for field and military use that deliver medicaments for the above conditions plus battlefield conditions such as exposure to chemical and biological weapons, battle fatigue, blood infections, sepsis, heat stroke, sun stroke, hypothermia, and fungal infections of the feet.

The following chart shows, for a number of essential oil actives, the local conditions (in the second column) that could be treated with an embodiment of the invention incorporating that essential oil, and (in the third column) the systemic conditions that could potentially be treated with an embodiment of the invention incorporating the oil as an active. It will be clear to one skilled in the art that the latter conditions are inherently more difficult targets for drug-delivery, but they are cited in the chart because of the high impact that a successful transdermal delivery system of the present invention would have.

Table showing a number of plant essential oils and the local and systemic conditions for which each essential oil may be used therapeutically.

| | Locally Treated | Systematic Conditions |
|---|---|---|
| Essential Oil | Conditions | Treated |
| Tea Tree Oil | Acne, Athlete's foot, | |
| | Boils, Burns, Ringworm, | |
| | lice, dandruff, colds, viral | |
| | infections, warts, rosacea, | |
| | Sinus infection, Earache. | |
| Melissa/lemon balm | | Sleep aid, mood elevator |
| Lavender | Heals burn wounds/sores, | Stress relief, calming, |
| | acne, insect bites, sores, | Alopecia (hair loss), |
| | sprains, hypertension, | rheumatism, vertigo |
| | rosacea | |
| Wintergreen Oil | Acne, Headache, Pain | Asthma, Arthritis |
| Goldenseal, garlic, turmeric, | Fungal infections | |
| yellow dock, sage, licorice, | | |
| black walnut, Calendula | | |
| officinalis, Pau d'Arco, | | |
| Myrrh, Peppermint, and | | |
| Oregano | | |
| Bergamot | Psoriasis, acne, abscesses, | Depression |
| | cold sores, cystitis, | |
| | eczema, warts | |
| Eucalyptus | Congestion, anti- | Poor Circulation, Asthma |
| | inflammatory, prevent | |
| | infection, anti-septic | |
| Frankincense | Skin Cancer, scars | Asthma |
| Chamomile Oil | Infections, acne, dry skin, | Allergies |
| | insect bites | |
| Lemon Oil | Cold Sores, insect bites | Cellulite |
| Lemongrass | Acne, athlete's foot, | Low Energy/Alertness |
| | muscle ache | |
| Orange Oil | | Insomnia, anxiety, nervous |
| | | tension, stress |
| Patchouli Oil | Eczema, cracked skin, | Low Libido |
| | scar tissue | |
| Angelica Root | Psoriasis | Toxin build up, stress |
| Anise | | Depression |
| Basil | Muscle aches | Memory, concentration |
| Bay | Dandruff | Poor circulation |
| Rosewood | Acne, stretch marks | |
| Cedarwood | Acne, dermatitis | Arthritis, anxiety |
| Clary sage | Labor pains | Asthma |
| Citronella | | Excessive perspiration |
| Coriander | nausea | |
| Cypress | Warts | Confidence, memory |
| Elemi | Wounds, cold sores | Grief |
| Fennel | | Water retention, toxin |
| | | buildup |
| Galbanum | Sores, stretch marks, lice, | |
| | cuts, boils, acne | |
| Geranium | Acne, lice | Menopause |
| Grapefruit | Water retention, toxin | |
| | build up, cellulite | |
| Helichrysum | Eczema, wounds | |
| Hyssop | Bruises | |
| Jasmine | Dry skin, labor pain | Depression, fatigue, |
| | | burnout, confidence, anger |
| Juniper Berry | | Gout, rheumatism, toxin |
| | | build up |
| Linden blossom | Wrinkles, acne, scars, oily | Insomnia, stress |
| | skin, dry skin | |
| Marjoram | | Rheumatism, arthritis |
| Melissa | Herpes, cold sores, | Insomnia |
| | shingles | |
| Myrtle | Acne, hemorrhoids, | Asthma, addiction |
| | ringworm, halitosis | |
| Neroli | scars | Depression, panic attacks |
| Niaouli | Acne | |
| Nutmeg | | Arthritis, poor circulation, |
| | | rheumatism |
| Parsley | Kidney infections and | |
| | stones | |
| Black pepper | Detox, cramps | Arthritis, poor circulation |
| Patchouli | Eczema, dandruff | |
| Petitgrain | | Rapid heartbeat, insomnia |
| Pine | Congestion | |
| Rose | Eczema | Depression, stress, |
| | | menopause |
| Rosemary | Muscle aches, dandruff | Arthritis, gout, exhaustion, |
| | | poor circulation, |
| | | rheumatism, neuralgia |
| Spearmint | Nausea, headache | Asthma |
| Sandalwood | Oily skin | Depression |
| Thyme | Dermatitis | Arthritis |
| Vetiver | Acne, muscle aches | Arthritis, rheumatism |
| Violet Leaf | | Insomnia, poor circulation, |
| | | shyness |
| Yarrow | Acne, hair care, | Arthritis, Insomnia, |
| | inflammation | hypertension, stress, tension |
| Ylang Ylang | Palpitations | Depression, Hypertension, |
| | | PMS symptoms, stress, |

Besides essential oils and the above-cited pain-relieving plant extracts, other plant extracts can be delivered to a mammal, particularly a human, with the use of this invention for other purposes. These include, but are by no means limited to:

Alopecia, baldness, etc. (applied via a cap, for example): Acacia bica, Terminalia bellirica, Terminalia chebula, Cocos nucifera, Sesamum indicum, Celastrus panniculatus, Myristica fragrans, Withenia somnifera, Sarvang asana, Padhastasna asana, Camelia sinensis, Serenoa repens;

Obesity, weight control, hyperlipidemia (e.g., applied via stomach-contacting fabric): Caralluma fibriata; Garcinia cambogia/hydroxycitric acid; Commiphora mukuk, Operculina turpethum, Acorns calamus, Boerhavia diffusa, Cyperus rotundus, Zingiber officinale, Piper nigrum, Plumbago zeylanica, Picrorhiza kurroa, Allium sativum, Piper longum, Embelia ribes;

Blood pressure control: Terminalia chebula, Terminalia balarica, Asphaltum, Emblica officinale, Tribulus terristris, Nerium indicum, Terminalia arjuna, Allium sativum;

Joint pain, osteoarthritis, rheumatoid arthritis (e.g., applied via a fabric contacting the sort joint(s)): Vitex negundo, Boswellia sacra, Cyperus scariozus, Boehavia diffusa, Hyoscyamus niger;

Muscle cramps: apple cider vinegar; ginger extract.

Vitality, sexual performance: Mucuna pruriens, Asparagus adscendens, Anacyclus pyrethrum, Coryophyllus aromaticus, Pueraria tuberose, Eulophia campestris;

Brain health, intellect: Bacopa Monniera, Convolvus pluricaulis, Withania somnifera, Ginkgo biloba, Celastrus panniculatus.

Stress-relieving: Sceletium tortuosum, various Salvia species.

Various aspects and embodiments will now be recited. These aspects and embodiments have been described and supported in further detail throughout the application and FIGURES.

Rod-Shaped Particles

In one aspect, a rod-shaped particle comprising an inner polymer matrix filled with dispersed solid active and partially skinned with a polymer that is substantially impermeable to the active is provided that conforms to the following mathematical conditions:

the ratio D/(K·u) is greater than 1;

the ratio 1K/D is less than 0.1;

the aspect ratio 1/d is between 1 and 50; and

where 1 is the length of the rod-shaped particle, d is the diameter of the rod, D is the diffusion constant and K the dissolution constant of the active in said inner polymer, and u=1 cm is a standard unit of length.

In one embodiment the ratio D/(K·u) is greater than 10. In one embodiment the ratio D/(K·u) is greater than 100.

In one embodiment the ratio 1K/D is less than 0.06.

In one embodiment the aspect ratio 1/d is between 2 and 20. In one embodiment the aspect ratio 1/d is between 2 and 10.

In one embodiment the area coverage of said inner polymer matrix by said skin is 80.0% to 99.9%. In one embodiment the area coverage of said inner polymer matrix by said skin is 85.0% to 99.5%. In one embodiment the area coverage of said inner polymer matrix by said skin is 90% to 99.0%.

In one embodiment said inner polymer matrix comprises one or more polymers selected from the group consisting of polysiloxanes (silicones), polyurethanes, polyanhydrides, polyisobutylene, elastin, natural rubber (polyisoprene), chloroprene, neoprene, butyl rubber, styrene-butadiene rubber (SBR), nitrile rubber, epichlorohydrin rubber, fluoroelastomers, polyether block amides, ethylene-vinylacetate (EVA), copolymers such as poly(styrene-b-isobutylene-b-styrene), ABS, styrenic block copolymers (TPE-s, such as Sofprene and Laprene), polyolefin blends (TPE-o), elastomeric alloys (TPE-v or TPV, such as Forprene), thermoplastic polyurethanes (TPU), thermoplastic copolyesters, and thermoplastic polyamides including Arnitel, Solprene, Engage, Hytrel, Dryflex and Mediprene, Kraton, and Pibiflex.

In one embodiment said inner polymer matrix comprises a crosslinked polymer selected from the group consisting of polyhydroxyethyl methacrylate (PolyHEMA), gelatin, starch derivatives, polyethylene glycol, celluloses, natural gums such as gum Arabic, gum tragacanth, xanthan gum, guar gum, gellan gum, and dextran.

In one embodiment said outer “skin” polymer is selected from the group consisting of polypropylene, polyvinyl chloride, PTFE (non-porous), polyvinylidene fluoride (PVDF), PMMA, shellac, polycarbonate (viz., Lexan), polybutylene terephthalate, epoxy, polyethylene terephthalate (PET), high-density polyethylene, celluloid, ABS, polyimide, nylon, phenol-formaldehyde resin, and polystyrene.

In one embodiment the length is greater than or equal to about 1 centimeter.

In one embodiment said active is selected from the group consisting of antimicrobials, antibiotics, antifungals, antiseptics, and astringents.

In one embodiment said active is selected from the group consisting of vasodilators, rubefacients, ceramide, emollients, dermoprotective, lipolytic, and epithelializing compound.

In one embodiment said active is a local anesthetic.

In one embodiment said active is an anti-inflammatory.

Fabric

In another aspect a fabric to which a multitude of particles as described herein are affixed is provided.

In one embodiment the fabric forms at least part of an article of clothing.

In one embodiment said article of clothing is selected from the group consisting of socks, hats, face/ski masks, scarves, tiaras, chokers, skullcaps, undergarments, skin guards, wrist bands, arm bands, knee pads, bras, nylon stockings, athletic supporters, robes, neck bands, head bands, ear muffs, gloves, diapers.

In one embodiment the fabric forms at least part of an article of bedding.

In one embodiment said article of bedding is selected from the group consisting of pillowcases, bedsheets, and blankets.

In one embodiment the fabric forms at least a part of a pillowcase.

Coatings

In another aspect, coating comprising a multitude of particles as described herein is provided.

In one embodiment the coating is in a form selected from the group consisting of paints, lacquers, varnishes, primers, stains, shellacs, sealants, and enamels.

Methods

In another aspect a method for treating a skin condition is provided comprising contacting areas of the skin of a mammal in need of treatment with a fabric to which a multitude of particles as described herein are affixed.

Sprays

In another aspect a spray is provided containing particles as described herein, which upon application to a substrate results in a multitude of said particles affixed to said substrate.

Materials

In one aspect, a material is provided comprising
a crosslinked polymer comprising N-isopropylacrylamide monomer units;
an active compound that is not water loaded within said crosslinked polymer; and
a plurality of solid platelets confined within said crosslinked polymer,

wherein said material is suitable for releasing said active into mammalian tissue.

In one embodiment said active compound is substantially contained within particles, said particles being in physical association with said crosslinked polymer.

In one embodiment said crosslinked polymer is swollen with water so as to form a hydrogel.

In one embodiment said hydrogel has a collapse temperature between about 25 and 50 degrees Centigrade.

In one embodiment said hydrogel has a collapse temperature between about 30 and 45 degrees Centigrade.

In one embodiment said hydrogel has a collapse temperature between about 33 and 40 degrees Centigrade.

In one embodiment a liquid which can be converted to the hydrogel by a chemical reaction is provided.

In one embodiment the material forms at least part of a contact lens.

In one embodiment said chemical reaction is a polymerization reaction.

In one embodiment said chemical reaction is a crosslinking reaction.

In one embodiment the release rate of active is substantially lower in water at 5 degrees Centigrade above said collapse temperature than at 5 degrees below said collapse temperature.

In one embodiment said crosslinked polymer is swollen with water so as to form a hydrogel.

In one embodiment said platelets and said particles are substantially confined within said hydrogel.

In one embodiment said platelets are derived from a clay.

In one embodiment said particles comprise reversed cubic phase or reversed hexagonal phase liquid crystalline matter.

In one embodiment said active is dispersed as a solid in said particles.

In one embodiment said particles comprise polymer of aqueous solubility less than or equal to about 1 mg/mL.

In one embodiment said polymer of low aqueous solubility is an elastomer at 25 degrees Centigrade.

In one embodiment said active is an active pharmaceutical ingredient.

In one embodiment said active is antimicrobial.

In one embodiment said active comprises a plant essential oil or a component thereof.

In one embodiment said active is configured to be delivered into tissue of a mammal when placed in contact with said tissue.

In one embodiment said platelets are characterized by average dimensions L×W×T, where L≧W>>T with T being the platelet thickness, and where the aspect ratio W/T is greater than or equal to about 3.

In one embodiment the aspect ratio is greater than or equal to about 5.

In one embodiment the aspect ratio is greater than or equal to about 10.

In one embodiment the average platelet thickness is less than or equal to about 1 micron.

In one embodiment the average platelet thickness is less than or equal to about 0.1 micron.

In one embodiment the average platelet thickness is less than or equal to about 10 nanometers.

In another aspect the material disclosed herein is affixed to a solid support.

In one embodiment said support is a fabric.

In one embodiment said fabric is an article of clothing.

In one embodiment said fabric is a pillowcase.

In one embodiment said fabric is a bed sheet.

In one embodiment said article of clothing is a sock.

In another aspect a material is provided consisting essentially of
a hydrogel comprising N-isopropylacrylamide monomer units;
one or more active compounds that are not water and are loaded within said hydrogel; and
a plurality of solid platelets confined within said hydrogel,

wherein said solid platelets are characterized by an average ratio of girth to thickness that is greater than or equal to about 10 to 1.

In another aspect a method for delivering an active which is not water into mammalian tissue is provided comprising

A. contacting said tissue with a material comprising
a crosslinked polymer comprising N-isopropylacrylamide monomer units;
an active compound that is not water loaded within said crosslinked polymer; and
a plurality of solid platelets confined within said crosslinked polymer, and

B. allowing said active to diffuse from said material to said tissue.

In another aspect a method for delivering a volatile active which is not water into the nasal passages of a human is provided comprising

A: placing in proximity to said nasal passages a material comprising
a crosslinked polymer comprising N-isopropylacrylamide monomer units;
said volatile active compound loaded within said crosslinked polymer; and
a plurality of solid platelets confined within said crosslinked polymer, and

B. allowing said active to diffuse from said material into said nasal passages.

In another aspect a method for delivering an active which is not water into tissue of a mammal in need of said active is provided comprising
contacting said tissue with a material comprising
a crosslinked polymer comprising N-isopropylacrylamide monomer units;
an active compound that is not water loaded within said crosslinked polymer; and
a plurality of solid platelets confined within said crosslinked polymer, and
allowing said active to diffuse from said hydrogel into said tissue.

In another aspect a material is provided comprising
a crosslinked polymer comprising N-isopropylacrylamide monomer units;
an active compound that is not water loaded within said crosslinked polymer; and
a plurality of solid platelets confined within said crosslinked polymer,

wherein said material is configured so as to deliver said active compound to a mammal in need of said active through the act of

i. contacting a tissue of said mammal with the material, and

ii. allowing said active compound to diffuse out of said material and to said mammalian tissue.

### EXAMPLES

The following examples illustrate the present invention but are not to be construed as limiting the invention.

### Example 1

An aqueous dispersion of cubic phase liquid crystalline microparticles containing the local anesthetic bupivacaine, at a particle concentration of 10 wt %, amounting to 5 mg/mL of bupivacaine free base, was prepared according to the methods described in detail in U.S. Pat. No. 7,713,440 to Anderson. The average microparticle size was approximately 0.5 microns. 1.23 grams of this dispersion was spiked with a trace amount (less than 1 mg) of the intensely-colored dye merocyanine 540.

A 30 wt % N-isopropylacrylamide monomer solution in water was prepared, and treated with alumina to remove MEHQ inhibitor. To 3.35 grams of this monomer solution were added 0.867 grams of the merocyanine-loaded dispersion of cubic phase particles, 0.305 grams of the crosslinker methylene-bis-acrylamide, 0.440 grams of Optigel powder, and approximately 0.01 grams of the secondary amine activator compound piperidine. After thoroughly mixing this to homogeneity, a solution of the initiator potassium persulfate in water (0.05 grams in approximately 0.3 mL water) was stirred into the mixture, and the contents sparged with nitrogen gas to remove oxygen. After 24 hours had passed, hydrogel had formed from the polymerization of the NIPAM and its crosslinking with the methylene-bis-acrylamide. During this 24 hour period, there were no visual signs of any separation or aggregation of the Optigel platelets. The resulting hydrogel was thus an embodiment of the present invention.

Exposure to ambient light, amplified by the oxidizing effect of the persulfate, caused photo-oxidation of the merocyanine 540. However, this oxidation is reversible, and color can be restored by treatment with a reducing agent, as seen below.

### Example 2

A portion of hydrogel from Example 1 was cut into two halves, both microwaved side by side for 30 seconds at 20% power, and then trimmed to the same weight. One portion was then placed in a test tube containing hot water, maintained at approximately 45-50 degrees C., and the other placed in a test tube containing water kept at ambient temperature, approximately 19 degrees C. The two hydrogel halves were treated as identically as possible at all times, except for the difference in temperature.

After 60 minutes, water was decanted off each of the two test tubes, allowed to equilibrate to ambient temperature, and then treated with 0.06 grams of the reducing agent stannous chloride. This reducing agent restored the color of the merocyanine, so that the strength of the color is a direct measure of the amount of merocyanine that was released by each hydrogel during the 60 minute period. As can be seen in FIG. 3, the color strength in the cold-water sample, labeled “C”, was strikingly more intense than the color in the hot-water sample, labeled “H”. This demonstrates that the release rate of dye from the hydrogel of this invention was much higher at 19 degrees C. than at elevated temperature (approx. 45 degrees C.). A visual estimate would be that the release rate at the lower temperature was at least 3 times higher than that at the hot temperature (200% higher).

### Example 3

The hydrogel from Example 2 is glued to a bamboo fabric using a polyurethane-based “Gorilla glue”, applied sparingly so as to avoid occluding the fabric. Curing of the glue results in a washable, local anesthetic-releasing patch of functionalized bamboo fabric, which has a higher rate of bupivacaine release at 30° C. than at 40° C. The fabric can be washed in a standard washing machine, at wash-cycle temperatures between 40° and 50° C., without unacceptable loss of active (bupivacaine).

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

The embodiments of the invention in which an exclusive property or privilege is claimed are defined as follows:

1. A rod-shaped particle comprising an inner polymer matrix filled with dispersed solid active and partially skinned with a polymer that is substantially impermeable to the active, conforming to the following mathematical conditions:
the ratio D/(K·u) is greater than 1;
the ratio 1K/D is less than 0.1;
the aspect ratio 1/d is between 1 and 50; and
where 1 is the length of the rod-shaped particle, d is the diameter of the rod, D is the diffusion constant and K the dissolution constant of the active in said inner polymer, and u=1 cm is a standard unit of length.

2. The particle of claim 1 in which the area coverage of said inner polymer matrix by said skin is 80.0% to 99.9%.

3. The particle of claim 1 in which said inner polymer matrix comprises one or more polymers selected from the group consisting of polysiloxanes (silicones), polyurethanes, polyanhydrides, polyisobutylene, elastin, natural rubber (polyisoprene), chloroprene, neoprene, butyl rubber, styrene-butadiene rubber (SBR), nitrile rubber, epichlorohydrin rubber, fluoroelastomers, polyether block amides, ethylene-vinylacetate (EVA), copolymers such as poly(styrene-b-isobutylene-b-styrene), ABS, styrenic block copolymers (TPE-s, such as Sofprene and Laprene), polyolefin blends (TPE-o), elastomeric alloys (TPE-v or TPV, such as Forprene), thermoplastic polyurethanes (TPU), thermoplastic copolyesters, and thermoplastic polyamides including Arnitel, Solprene, Engage, Hytrel, Dryflex and Mediprene, Kraton, and Pibiflex.

4. The particle of claim 1 in which said inner polymer matrix comprises a crosslinked polymer selected from the group consisting of polyhydroxyethyl methacrylate (PolyHEMA), gelatin, starch derivatives, polyethylene glycol, celluloses, natural gums such as gum Arabic, gum tragacanth, xanthan gum, guar gum, gellan gum, and dextran.

5. The particle of claim 1 in which said outer “skin” polymer is selected from the group consisting of polypropylene, polyvinyl chloride, PTFE (non-porous), polyvinylidene fluoride (PVDF), PMMA, shellac, polycarbonate (viz., Lexan), polybutylene terephthalate, epoxy, polyethylene terephthalate (PET), high-density polyethylene, celluloid, ABS, polyimide, nylon, phenol-formaldehyde resin, and polystyrene.

6. The particle of claim 1 in which the length is greater than or equal to about 1 centimeter.

7. The particle of claim 1 in which said active is selected from the group consisting of antimicrobials, antibiotics, antifungals, antiseptics, and astringents.

8. The particle of claim 1 in which said active is selected from the group consisting of vasodilators, rubefacients, ceramide, emollients, dermoprotective, lipolytic, and epithelializing compound.

9. The particle of claim 1 in which said active is a local anesthetic.

10. The particle of claim 1 in which said active is an anti-inflammatory.

11. A fabric to which a multitude of particles according to claim 1 are affixed.

12. The fabric of claim 11, wherein the fabric forms at least part of an article of clothing.

13. The fabric of claim 12 in which said article of clothing is selected from the group consisting of socks, hats, face/ski masks, scarves, tiaras, chokers, skullcaps, undergarments, skin guards, wrist bands, arm bands, knee pads, bras, nylon stockings, athletic supporters, robes, neck bands, head bands, ear muffs, gloves, and diapers.

14. The fabric of claim 11, wherein the fabric forms at least part of an article of bedding.

15. The fabric of claim 14 in which said article of bedding is selected from the group consisting of pillowcases, bedsheets, and blankets.

16. The fabric of claim 11, wherein the fabric forms at least a part of a pillowcase.

17. A coating comprising a multitude of particles according to claim 1.

18. The coating of claim 17, wherein the coating is in a form selected from the group consisting of paints, lacquers, varnishes, primers, stains, shellacs, sealants, and enamels.

19. A method for treating a skin condition comprising contacting areas of the skin of a mammal in need of treatment with a fabric to which a multitude of particles according to claim 1 are affixed.

20. A spray containing particles according to claim 1, which upon application to a substrate results in a multitude of said particles affixed to said substrate.
